(19) United States
(12) Patent Application Publication
(10) Pub. No.: US 2023/0372464 A1
(43) Pub. Date: Nov. 23, 2023

(54) CHOLERA VACCINE FORMULATION

(71) Applicant: VALNEVA SWEDEN AB, Solna (SE)

(72) Inventors: Valentina Screpanti-Sundquist, Spånga (SE); Janet Hoogstraate, Uddinge (SE); Fredrik Bylund, Stockholm (SE)

(73) Assignee: VALNEVA SWEDEN AB, Solna (SE)

(21) Appl. No.: 18/027,657

(22) PCT Filed: Oct. 6, 2021
(86) PCT No.: PCT/EP2021/077576

§ 371 Date: Mar. 22, 2023

(30) Foreign Application Priority Data
Oct. 7, 2020 (EP) EP20200560.9

(51) Int. Cl. A61K 39/02; A61P 31/04

(52) U.S. Cl. CPC A61K 39/107; A61P 31/04; A61K 2039/521

(57) ABSTRACT

Described herein are dry compositions that can be stored at ambient temperature without major loss of potency.

### FIELD OF THE INVENTION

The present invention relates to room temperature stable pharmaceutical compositions and to processes of manufacturing such compositions.

### BACKGROUND

The majority of marketed vaccines are not stable enough and therefore have to be kept frozen or refrigerated during long-term storage in order to maintain their potency (see Chen & Kristensen 2009; Kumru et al. 2014). Even existing dry vaccines generally require storage at low temperatures, i.e. between 2° C. to 8° C. (see Kumru et al. 2014). Cold-chain dependency makes vaccines susceptible to damage and ineffectiveness especially in low-income countries. Also stringent requirement of cold conditions for shipping and storage can make non-stable vaccines unavailable to some categories of populations of poor countries due to unreliable transportation system. In contrast, vaccines that are stable at ambient temperatures and do not need refrigerated storage conditions have significant advantages for shipment and stockpiling. Consequently, developing thermostable vaccine formulations and reducing their dependency on the cold chain could have great economic and health benefits.

A common approach applied to produce a thermostable pharmaceutical composition including a vaccine is drying of liquid ingredients to a state with low water content and/or water activity. Dry vaccine formulations are generally less sensitive to temperature-induced degradation.

Several methods are available for preparing dry vaccines. Freeze drying (lyophilization), a traditional method for drying proteins, is also used for manufacturing dry vaccines. It involves freezing of a liquid solution followed by removal of water by sublimation of ice and thereafter by desorption of remaining water at low pressure and higher temperature. This results in a dried cake in the final container and requires reconstitution before administration.

Although lyophilization technology has resulted in the development of many successful live, attenuated viral and bacterial vaccines, most of these vaccines still require storage at 2-8° C. or below. In some cases, lyophilization leads to significant damage of a vaccine, e.g. a measles virus vaccine (see Ohtake et al. 2010). Examples of lyophilized vaccines that have to be stored at refrigerated conditions are: Hiberix® (GSK), Rotarix® (GSK), Imovax® (Sanofi Pasteur), YF-Vax® (Sanofi Pasteur), JE-Vax® (Osaka), M-M-RVAXPRO® (Merck) and others.

Currently there are three inactivated cholera vaccines available at the market. WC-rBS marketed as Dukoral® (Valneva Sweeden AB) is a monovalent inactivated vaccine containing killed whole cells of V. cholerae O1 plus additional recombinant cholera toxin B subunit. BivWC marketed as “Shanchol™” (Sanofi Pasteur, India), Euvichol® (Eubiologics, Republic of Korea) and “mORC-VAX” (Vabiotech, Vietnam) is a bivalent inactivated vaccine containing killed whole cells of V. cholerae O1 and V. cholerae O139. mORC-VAX is only available in Vietnam. All three vaccines are in the liquid form and require storage at refrigerated temperature 2° C. to 8° C.

Dukoral® is a suspension taken orally with bicarbonate buffer, which protects the antigens from the gastric acid. The anti-toxin intestinal antibodies prevent the cholera toxin from binding to the intestinal mucosal surface, thereby preventing the toxin-mediated diarrhoeal symptoms (Holmgren et al. 1989 “Oral immunization against cholera.” Current topics in Microbiology and Immunology, Vol. 146, p. 197). Dukoral® can be given to all individuals over the age of 2 years. There must be a minimum of 7 days, and no more than 6 weeks, delay between each dose. Children aged 2-5 require a third dose. Dukoral® is mainly used for travellers. Two doses of Dukoral® provide protection against cholera for 2 years. Other three marketed vaccine do not require a buffer solution for administration. They are given to all individuals over the age of one year. There must be a minimum of two weeks delay between each dose of these vaccines. Two doses of Shanchol™ and Euvichol® provide protection against cholera for 3 years, while a single dose provides short term protection.

The only one lyophilized live attenuated cholera vaccine, named CVD 103-HgR or Vaxchora® (PaxVax, USA), was approved by the US FDA. Vaxchora® is an oral vaccine composed of V. cholerae CVD 103-HgR constructed from the serogroup O1 classical Inaba strain by deleting the catalytic domain sequence of both copies of the ctxA gene, which prevents the synthesis of active cholera toxin (CT). This attenuated strain remains able to synthesize the immunogenic non-toxic B subunit of cholera toxin encoded by the ctxB gene (Chen et al., 2016). Required storage temperature for Vaxchora® is 2° C. to 8° C. (in EU) or −25° C. to −15° C. (in US).

Previous attempts to develop a dry cholera vaccine formulation by freeze-drying and using 25 mg/ml sucrose or trehalose as stabilizer have not resulted in obtaining a commercial product; also no data on long-term stability at ambient temperature were reported (Borde A, Larsson A, Holmgren J, Nygren E. 2011. “Preparation and evaluation of a freeze-dried oral killed cholera vaccine formulation”. Eur J Pharm Biopharm. 79(3):508-18).

Consequently, there is a need for thermostable formulations of existing or novel vaccines in order to reduce or eliminate dependency on the cold chain. Particularly, production of the cold chain-free cholera vaccine is highly desirable.

### SUMMARY OF THE INVENTION

The present invention provides thermostable pharmaceutical compositions, especially vaccines, and methods for preserving them from degradation at ambient temperatures. These methods include processes of preparing dry formulations of marketed liquid pharmaceutical compositions, including vaccines, or developing novel dry compositions. Dry compositions of the inventions have low water activity (about 0.15 or less) and therefore remain stable at room or elevated temperature up to 40° C. for extended period of time. Thus, by producing stable dry formulations, shelf lives of the pharmaceutical composition can be sufficiently prolonged and requirement of cold chain can be eliminated.

The pharmaceutical composition of the invention usually comprises a bioactive material, such as a microorganism and/or its subunit(s), a stabilizing agent and, optionally, a protective (preservative) agent. In some embodiments, the biological material of the pharmaceutical composition comprises bacteria, or virus, or isolated protein(s), or recombinant protein(s), or polypeptide(s), or nucleic acid(s), or polysaccharide(s), or lipid(s), or toxin(s), and/or various combinations thereof.

In some embodiments, the pharmaceutical composition of the invention comprises bacteria selected from, but not limited to, the group consisting of Vibrion cholerae, Clostridium difficile, Clostridium perfringens, Clostridium botulinum, Clostridium tetani, Corynebacterium diphtheria, Shigella dysentheriae, Staphylococcus aureus, Pseudomonas aeruginosa, Bordetella pertussis, Bacillus anthracis and Escherichia coli. In a preferred embodiment, the composition of the invention comprises Vibrio cholerae. In some embodiments, the bacteria are live attenuated or inactivated (killed) bacteria. In some embodiments, the composition comprises whole-cell bacteria.

In other embodiments, the pharmaceutical composition of the invention comprises a combination of whole-cell bacteria and a bacterial toxin. In some embodiments, the composition of the invention comprises at least one bacterial toxin selected from, but not limited to, the group consisting of cholera toxin, staphylococcal toxins, diphtheria toxin, tetanus toxin, pertussis toxin, shiga toxin, shiga-like toxin, botulinum neurotoxin, Clostridium difficile toxins, Clostridium perfringens alpha toxin, Bacillus anthracis toxin, Pseudomonas aeruginosa alpha toxin, heat-labile enterotoxin (LT) of enterotoxigenic Escherichia coli (ETEC) and heat-stable enterotoxin (ST) of enterotoxigenic Escherichia coli (ETEC). Toxins of the compositions may be naturally isolated toxins, recombinant toxins, modified toxins, or toxin subunits.

In addition, the pharmaceutical composition of the invention comprises a pharmaceutically acceptable carrier and/or excipient. The appropriated carrier or excipient may be selected from, but not limited to, a buffer, diluent, stabilizer, preservative, surfactant, etc. either alone or in combinations.

Usually, stability of the pharmaceutical composition comprising a biological material is improved in the presence of a stabilizer in which the biological material is embedded. In some embodiments, the composition of the invention comprises a sufficient amount of at least one stabilizing agent. Examples of stabilizing agents include, but are not limited to, human and bovine serum albumin, egg albumin, gelatin, immunoglobulin, skim milk powder, casein, soya protein, wheat protein and any protein hydrolysates, carbohydrates including monosaccharides (e.g. galactose, mannose, sorbose, etc.), disaccharides (e.g., sucrose, trehalose, lactose, etc.), polysaccharides (e.g., dextran, maltodextrin), amino acid (e.g., leucine, lysine, alanine, arginine, histidine, glutamate, etc.), methylamine such as betaine), polyol such as sugar alcohol (e.g. glycerin, glycerol, sorbitol, arabitol, erythitol, mannitol, etc.), synthetic polymers such as propylene glycol, polyethylene glycol, polyvinylpyrrolidone, pluronics, etc. Preferably, the stabilizer is a sugar stabilizer such as sucrose, tregalose, raffinose, lactose, maltose, mannitol, sorbitol, xylitol, maltodextrin, or variable combinations thereof. More preferably, the sugar stabilizer is sucrose or maltodextrin, or a combination of both. Particularly, the composition may comprise from 10 to 100 mg/mL sucrose.

The composition of the invention is prepared in dry form. According to the method of preparation, the composition may be a freeze-dried (lyophilized), spray-dried, foam dried, or alike. The dry composition of the invention has a residual moisture content (residual water) about or less than 3%, particularly between about 3% and 1% (Mensink et al. 2017. “How sugars protect proteins in the solid state and during drying (review): Mechanisms of stabilization in relation to stress conditions.” Eur J Pharm Biopharm. 114, 288-295), preferably between about 3% and 2%. The dry composition may be formulated in dosage units as a powder, tablets, granules or capsules.

In a particular embodiment, the dry composition of the invention has a water activity equal to or less than 0.15, preferably between 0.1 and 0.02, particularly about 0.1, 0.09, 0.08, 0.07, 0.06, 0.05, 0.04 or 0.03.

Generally, the dry composition of the invention is stable inside the temperature range of about 20° C. to 40° C., especially about 25° C. to 35° C., preferably about 25° C. or 30° C. for at least one year, preferably at least 2 or 3 years, even more preferably up to 5 years without significant drop of potency. Please note that depending on the region, WHO recommends room temperature storage to be defined as either 25° C. or 30° C. at relative humidity 60±5% or 75±5% as e.g. is defined for climatic zone IV (WHO_Annex Technical Report Series, No. 863, 1996). In a certain embodiment, the composition that has a water activity equal to or less than 0.15 is stable inside the temperature range of about 20° C. to 40° C., especially about 20° C. to 35° C., preferably about 25° C. or 30° C. for at least one year, preferably at least 2 or 3 years, even more preferably up to 5 years.

In a particular embodiment, the composition that has a water activity equal to or less than 0.15 has prolonged storage life at room temperature or elevated temperature as compared to a composition that has a water activity of more than 0.15. In more particular embodiment, the composition that has a water activity equal to or less than 0.1 has prolonged storage life at room temperature or elevated temperature as compared to a composition that has a water activity of more than 0.1.

In a preferred embodiment, potency of the composition that has a water activity equal to or less than 0.15 does not deviate more than +1-50% as compared to the same composition having a water activity more than 0.15 upon storage at the given conditions. In more preferred embodiment, potency of the composition that has a water activity equal to or less than 0.15 does not deviate more than +/−30% as compared to the same composition having a water activity more than 0.15 upon storage at the given conditions. In one particular embodiment, the pharmaceutical composition of the invention is a vaccine, especially a dry formulation of a vaccine. Additionally, the vaccine may be a whole-cell vaccine, a subunit vaccine, a bacterial vaccine, a viral vaccine, a VLP vaccine, a protein or (poly)peptide vaccine, a polysaccharide-conjugated vaccine or lipid-conjugated vaccine. Particularly, the vaccine of the invention may be a live attenuated or inactivated whole-cell vaccine. The vaccine may be a mono-, bi-, or multivalent vaccine. Furthermore, the vaccine of the invention may be admixed with an adjuvant. The vaccine may elicit a systemic and/or mucosal immune response.

In a more particular embodiment, the composition of the present invention is a dry cholera vaccine formulation comprising V. cholerae bacteria.

In some particular embodiments, the dry cholera vaccine formulation comprises at least one V. cholerae strain selected from V. cholerae O1 Inaba classical biotype, V. cholerae O1 Inaba E1 Tor biotype and V. cholerae O1 Ogawa classical biotype. In a preferred embodiment, the dry cholera vaccine formulation comprises three bacterial strains: V. cholerae O1 Inaba classical biotype, V. cholerae O1 Inaba E1 Tor biotype, V. cholerae O1 Ogawa classical biotype. In additional embodiment, the dry cholera vaccine comprises heat and/or formalin inactivated V. cholerae bacteria.

In some embodiments, bacteria titer in the dry vaccine formulation is between 10⁵ and 10¹⁵ total V. cholerae cells per dosage, preferably between 10⁸ and 10¹² total V. cholerae cells per dosage, more preferably between 10¹¹ and 10¹² total V. cholerae cells per dosage. In one particularly preferred embodiment, the vaccine contains between approximately 1.0×10¹¹ and 1.5×10¹¹ total V. cholerae cells per dosage. In another particularly preferred embodiment, the vaccine contains approximately 1.25×10¹¹ total V. cholerae cells per dosage.

In still one embodiment, the dry cholera vaccine may further comprises a recombinant cholera toxin (CT) or its B subunit (CTB). The amount of the recombinant CTB (rCTB) is between about 0.1 mg and 10 mg, preferably between 0.75 and 1.5 mg, more preferably about 1 mg per the vaccine dose.

In one preferred embodiment, the dry vaccine comprises per dose between about 1.0×10¹¹ and 1.5×10¹¹, preferably about 1.25×10¹¹ total amount of bacteria of the following strains:
Vibrio cholerae O1 Inaba, classical biotype (heat inactivated),
Vibrio cholerae O1 Inaba, El Tor biotype (formalin inactivated),
Vibrio cholerae O1 Ogawa, classical biotype (heat inactivated),
Vibrio cholerae O1 Ogawa, classical biotype (formalin inactivated),
   excipients such as sodium dihydrogen phosphate monohydrate (2.0 mg), disodium hydrogen phosphate dihydrate (9.4 mg) and sodium chloride (26 mg), further comprising a stabilizer, and wherein said vaccine has a water activity of less than or equal to 0.15.

In another preferred embodiment, the dry vaccine comprises per dose between about 1.0×10¹¹ and 1.5×10¹¹, preferably about 1.25×10¹¹ total amount of bacteria of the following strains:
Vibrio cholerae O1 Inaba, classical biotype (heat inactivated),
Vibrio cholerae O1 Inaba, El Tor biotype (formalin inactivated),
Vibrio cholerae O1 Ogawa, classical biotype (heat inactivated),
Vibrio cholerae O1 Ogawa, classical biotype (formalin inactivated),
   a recombinant cholera toxin B subunit (rCTB) (0.75 to 1.0 mg), and
   excipients such as sodium dihydrogen phosphate dihydrate (2.0 mg), disodium hydrogen phosphate dihydrate (9.4 mg), sodium chloride (26 mg), further comprising a stabilizer, and wherein said vaccine has a water activity of less than or equal to 0.15.

In one more preferred embodiment, the stabilizer of the dry cholera vaccine formulation is a sugar, particularly maltodextrin or sucrose, or both combined respectively in ratio 9:1 (w/w) or 4:1 (w/w).

The dry cholera vaccine composition remains stable inside the temperature range of 20° C. to 40° C., especially at about 25° C. for at least two years, preferably more than two years, and its potency does not deviate more than +/−50% upon storage at the given conditions.

The present invention also provides use of the dry vaccine formulation for prevention and/or treatment of a bacterial or viral infection and/or an associated disease. Such dry vaccine may be reconstituted in water or buffer and then administered to a subject by one of the acceptable routes, e.g. orally, intramuscularly, intravenously, intradermally, intracutaneously, subcutaneously, bucally, or parenterally. Alternatively, the vaccine may be applied in dry form, via digestive (orally) or respiratory (by inhalation) route. In some embodiments, the vaccine may be administered to a subject as a single dose, or as a multiple dose, although as a booster.

The present invention also includes a use of the dry V. cholerae vaccine for treating and/or preventing V. cholerae infection and/or cholera disease.

The present invention also includes the method for treating and/or preventing V. cholerae infection and/or cholera disease, which comprises administering to a subject a therapeutically effective amount of the dry V. cholerae vaccine.

Additionally, the present invention provides methods (processes) for producing dry pharmaceutical compositions, including dry vaccine formulations, that comprise freeze drying, or spray drying or any modification thereof.

This invention is not limited in its application to the details of construction and the arrangement of components set forth in the following description or illustrated in the drawings. The invention is capable of other embodiments and of being practiced or of being carried out in various ways.

### BRIEF DESCRIPTION OF THE DRAWINGS

The accompanying drawings are not intended to be drawn to scale. For purposes of clarity, not every component may be labeled in every drawing. In the drawings:

FIG. 1. Particle size distribution of pure Dukoral® vaccine suspension and after addition of excipients: A—liquid composition with marltodextrin; B—liquid composition with marltodextrin:sucrose in ration 9:1; C—liquid composition with marltodextrin:sucrose in ration 4:1.

FIG. 2. Particle size distribution of pure Dukoral® vaccine suspension (red), liquid composition A with maltodextrin (green), re-hydrated spray-dried powder of composition A (blue) and re-hydrated freeze-dried powder of composition A (purple).

FIG. 3. Particle size distribution of pure Dukoral® vaccine suspension (red), liquid composition B with maltodextrin (green), re-hydrated spray-dried powder of composition B (blue) and re-hydrated freeze-dried powder of composition B (purple).

FIG. 4. Particle size distribution of pure Dukoral® vaccine suspension (red), liquid composition C with maltodextrin (green), re-hydrated spray-dried powder of composition C (blue) and re-hydrated freeze-dried powder of composition C (purple).

FIG. 5. Light microscopy images of (A) pure Dukoral® vaccine; (B) liquid composition A with maltodextrin; (C) liquid composition B with marltodextrin:sucrose in ration 9:1; (D) liquid composition C with marltodextrin:sucrose in ration 4:1. Images were acquired using a 100X_{Oil} objective.

FIG. 6. Light microscopy images of re-hydrated powders of (A) freeze-dried composition A; (B) freeze-dried composition B; (C) freeze-dried composition C; (D) spay-dried composition A; (E) spray-dried composition B; (F) spray-dried composition C. Images were acquired using a 100X_{Oil} objective.

FIG. 7. Stability of dried Dukoral® samples vs. pure Dukoral® suspension stored at 5° C. (A) Stability determined by LPS assay; (B) stability determined by Mancini test; (C) stability determined by absorbance at 600 nm.

FIG. 8. Stability of dried Dukoral® samples vs. Dukoral® suspension stored at 25° C. (A) Stability determined by LPS assay; (B) stability determined by Mancini test; (C) stability determined by absorbance at 600 nm.

FIG. 9. Stability of dried Dukoral® samples vs. Dukoral® suspension stored at 40° C. (A) Stability determined by LPS assay; (B) stability determined by Mancini test; (C) stability determined by absorbance at 600 nm.

### DETAILED DESCRIPTION OF THE INVENTION

### Definitions

“Dosage form” is a specific mixture of drug substances (active pharmaceutical ingredients) and inactive components (excipients) presented in a particular configuration to facilitate easy and accurate administration and delivery of active drug substances.

“Efficacy” is maximal effect a pharmaceutical composition (vaccine) can produce. Efficacious vaccine can have high or low potency.

“Potency” is amount of a pharmaceutical composition (vaccine dose) needed for a given effect.

“Shelf life” or “storage life” is a period of time during which a vaccine is expected to comply with the specification as determined by stability studies. Shelf life is used for the final product; storage period is used for the intermediates (WHO_TRS 962, Annex 5).

“Stability” is the ability of a composition to retain its chemical, physical, biological and/or immunological properties within specified limits upon storage. Stability can be measured at a selected temperature and humidity conditions for a selected time period.

“Thermal stability” is stability of a vaccine after exposure to a temperature higher than that recommended for storage for a specified period of time often expressed in terms of change in potency.

“Storage temperature ranges”: room temperature is between 15° C. and 25° C. (59° F. and 77° F.); elevated temperature is above 25° C., up to 40° C. (104° F.); cool temperature means between 8° C. and 15° C. (46° F. and 59° F.); refrigerator or cold temperature is between 2° C. and 8° C. (36° F. and 46° F.); freezer temperature is between −50° C. and −15° C. (−58° F. and +5° F.).

“Relative Humidity” or RH in the context of storage stability refers to the amount of water vapor in the air at a given temperature. Relative humidity is usually less than that required to saturate the air and is expressed in percent of saturation humidity.

“Water activity” or A_{w} is defined as the vapor pressure of water above a sample divided by that of pure water at the same temperature. Pure distilled water has a water activity of exactly one.

The present disclosure includes pharmaceutical compositions as defined in claims 1 to 31 and methods as defined in claims 32-34. The compositions and methods provided herein solve the problem of producing thermostable compositions containing bioactive materials, especially vaccines, with a significantly extended lifetime and cold-chain free storage.

The biological material of the compositions described herein may be whole-cell bacteria or their subunits, viruses or viral particles, proteins or polypeptides, nucleic acids, polysaccharides, lipids, hormones, toxins, protein conjugates and various combinations thereof. In some embodiments, the biological material may be an intact natural product or isolated from a natural source. In some embodiments, the biological material may be produced by recombinant techniques.

In some embodiments, the composition comprises a virus selected from, but not limited to, the group consisting of Adenovirus, Chikungunia virus, Coronavirus, SARS-CoV2, Cytomegalovirus, Dengue virus, Epstain-Barr virus, Ebola virus, Enterovirus, Influenza virus, Japanese Encephalitis virus, Hepatitis A virus, Hepatitis B virus, Hepatitis C virus, human Immunodeficiency virus, human papilloma virus, Herpes Simplex virus, Herpes Zoster virus, human Methapneumovirus, human rhinovirus, Measles virus, Mumps virus, paramyxovirus, Parvovirus B19, polyovirus, human parainfluenza virus, Rabies virus, Respiratory Syncytial virus, Rubella virus, Rotavirus, Smallpox virus, tick borne encephalitis virus, Varicella-zoster virus, Vaccinia virus, West Nile virus, Yellow Fever virus, and Zika virus.

In some embodiments, the virus of the composition thereby may be naturally isolated virus (natural isolate), modified virus (mutant), recombinant virus or virus vector. In some embodiments, the composition may comprise a combination of different isolates of the same virus species or different virus variants. In some embodiments, the composition may comprises live, attenuated virus or inactivated (killed) virus. In some embodiments, the composition may comprises an entire virion, a virus like particle, a viral DNA or RNA, vector that encode one or more viral protein(s), chimeric virus and/or the like.

In other embodiments, the composition of the invention comprises a bacteria selected from, but not limited to, the group consisting of Bacillus anthracis, Bordetella bronchiceptica, Bordetella pertussis, Borrelia burgdorferi, Brucella abortis, Brucella species, Candida albicans, Chlamydia pneumonia, Chlamidia trachomatis, Chlamidia psittaci, Clostridium difficile, Clostridium perfringens, Clostridium botulinum, Clostridium tetani, Corynebacterium diphtheria, Enterococcus faecalis, Enterobacter species, Escherichia coli, Helicobacter pylori, Haemophilus influenza, Klebsiella pneumohiae, Legionella pneumophila, Leishmania species, Listeria monocytogenes, Mycobacterium leprae, Mycobacterium tuberculosis, Mycoplasma species, Niesseria meningitides, Niesseria gonorrhoeae, Pseudomonas aeruginosa, Salmonella thyphimurium, Shigella dysentheriae, Shigella shinga, Staphylococcus aureus, Staphylococcus epidermidis, Streptococcus pneumoniae, Streptococcus agalactiae, Streptococcus pyogenes, Vibrio cholerae. Vibrio parahaemolyticus, Yersinia entercolitica, and Yersinia pestis.

In some embodiments, the bacteria of the composition described herein may be natural isolates, modified variants (mutants) or recombinantly produced bacteria. In some embodiments, the bacteria may be live attenuated or inactivated (killed) bacteria. In some embodiments, the composition may comprise bacteria of one strain or combination of different strains/clinical isolates of the same or different species.

In some embodiments, the composition may comprises live, attenuated virus or inactivated (killed) virus. In other embodiments, the composition may comprises whole cell bacteria, disintegrated bacterial cells, bacterial cell fragments, bacterial protein(s), bacterial DNA or RNA, bacterial membranes, bacterial lipid(s), bacterial polysaccharides, bacterial toxin(s), and/or different variants and combinations thereof.

In some embodiments, the composition of the invention comprises a bacterial proteinaceous toxin (protein toxin) selected from, but not limited to, the group consisting of cholera (Vibrio cholerae) toxin, staphylococcal toxins, diphtheria toxin, tetanus toxin, pertussis toxin, shiga toxin, shiga-like toxin, botulinum neurotoxin, Clostridium difficile toxins, Clostridium perfringens alpha toxin, Bacillus anthracis (anthrax) toxin, Pseudomonas aeruginosa alpha toxin, heat-labile enterotoxin (LT) of enterotoxigenic Escherichia coli (ETEC) and heat-stable enterotoxin (ST) of enterotoxigenic Escherichia coli (ETEC).

In some embodiments, the composition of the invention comprises a part (or fragment) of a bacterial toxin, such as a bacterial toxin subunit. Many protein toxins consists of two components, a subunit A which is responsible for the enzymatic activity of the toxin and such a subunit B which is non-toxic and concerned with binding to a specific receptor on the host cell membrane. In some embodiments, the composition of the invention comprises a non-toxic B subunit of a bacterial toxin, e.g. B subunit of cholera toxin (CTB), B subunit of diphtheria toxin, B subunit of pertussis toxin, B subunit of shiga toxin, B subunit of botulinum toxin, B subunit of anthrax toxin, B subunit of Bordetella pertussis AC toxin, B subunit of E. coli heat labile toxin LT, B subunit of Pseudomonas exotoxin A and Staphylococcus aureus exfoliatin B. Alternatively, the toxin (or toxin fragment, subunit) can be used in detoxified form (toxoid) which retain its antigenicity and immunizing capacity. Toxoids can be obtained by treating toxins with reagents such as formalin, iodine, pepsin, ascorbic acid, ketones, etc.

Vibrio cholerae Compositions

In a particular embodiment, the composition of the present invention comprises bacteria of V. cholerae sp. As described herein, a V. cholerae is a Gram-negative, curved rod-shaped bacterium with a polar flagellum. It is a facultative anaerobe and tends to tolerate alkaline media but is sensitive to acid (Finkelstein, Medical Microbiology “Cholera, Vibrio cholerae O1 and O139, and other Pathogenic Vibrios; 4^{th} Edition U.T. Medical Branch at Galveston (1996)).

V. cholerae are classified into distinct groups based on the structure of the O antigen of the LPS. In general, V. cholerae strains are classified as serogroup O1, serogroup O139, or non-O1/non-O139 based on agglutination of the bacterial cells (or lack thereof) in O1 and/or O139 antiserum. The non-O1/non-O139 strains have been divided into groups O2 through O138 based on the lipopolysaccharide (LPS) somatic (O) antigen. The majority of non-O1/non-O139 strains are not associated with cholera disease.

In one embodiment, the V. cholerae strain is V. cholerae O1. In yet one embodiments, the V. cholerae strain is V. cholerae O139. In another embodiment, the V. cholerae belongs to a non-O1 serogroup. Examples of non-O1 serogroups include the O2, O3, O4, O5, O6, O7, O8, O9, O10, O11, O12, O13, O14, O15, O16, O17, O18, O19, O20, O21, O22, O23, O24, O25, O26, O27, O28, O29, O30, O31, O32, O33, O34, O35, O36, O37, O38, O39, O40, O41, O42, O43, O44, O45, O46, O47, O48, O49, O50, O51, O52, O53, O54, O55, O56, O57, O58, O59, O60, O61, O62, O63, O64, O65, O66, O67, O68, O69, O70, O71, O72, O73, O74, O75, O76, O77, O78, O79, O80, O81, O82, O83, O84, O85, O86, O87, O88, O89, O90, O91, O92, O93, O94, O95, O96, O97, O98, O99, O100, O101, O102, O103, O104, O105, O106, O107, O108, O109, O110, O111, O112, O113, O114, O115, O116, O117, O118, O119, O120, O121, O122, O123, O124, O125, O126, O127, O128, O129, O130, O131, O132, O133, O134, O135, O136, O137, and O138 groups.

In yet another embodiment, the composition described herein may contain strains of V. cholerae belonging to different O groups. In still another embodiment, the composition may comprise one or more strains of V. cholerae O1 and one or more strains of V. cholerae belonging another O group.

The V. cholerae O1 group contains two major biotypes, El Tor and classical, each of which can be further distinguished into three serotypes based on the composition of the O antigen: Inaba, Ogawa, and Hikojima. Bacterial cells of each of the serotypes express the common “A” antigen; cells of the Ogawa serotype also express the “B” antigen i.e. express A+B antigens; cells of the Inaba serotype also express the “C” antigen, i.e. express A+C antigens; and cells of the Hikojima serotype express also the “B” and “C” antigens, i.e. express A+B+C antigens.

In some embodiments, the composition described herein comprises at least one (e.g., 1, 2, 3, 4, 5, or more) strain belonging to V. cholerae O1 El Tor biotype. In some embodiments, the composition described herein comprises at least one (e.g., 1, 2, 3, 4, 5, or more) strains belonging to V. cholerae O1 classical biotype. In some embodiments, the composition described herein comprises at least one (e.g., 1, 2, 3, 4, 5, or more) strains belonging to V. cholerae O1 El Tor biotype and at least one (e.g., 1, 2, 3, 4, 5, or more) strains belonging to V. cholerae O1 classical biotype. In some embodiments, the composition described herein comprises at least one (e.g., 1, 2, 3, 4, 5, or more) strain belonging to V. cholerae O1 El Tor biotype. In some embodiments, the compositions described herein comprise at least one (e.g., 1, 2, 3, 4, 5, or more) strains belonging to V. cholerae O1 classical Hikojima biotype. In some embodiments, the compositions described herein comprise at least one (e.g., 1, 2, 3, 4, 5, or more) strains belonging to V. cholerae O1 El Tor Hikojima biotype.

In some embodiments, the composition described herein comprises a combination of at least one (e.g., 1, 2, 3, 4, 5, or more) strain belonging to V. cholerae O1 El Tor biotype and cholera toxin. In some embodiments, the composition described herein comprises a combination of at least one (e.g., 1, 2, 3, 4, 5, or more) strains belonging to V. cholerae O1 classical biotype and cholera toxin. In some embodiments, the composition described herein comprises a combination of at least one (e.g., 1, 2, 3, 4, 5, or more) strains belonging to V. cholerae O1 El Tor biotype and at least one (e.g., 1, 2, 3, 4, 5, or more) strains belonging to V. cholerae O1 classical biotype and cholera toxin. In some embodiments, the composition described herein comprises a combination of at least one (e.g., 1, 2, 3, 4, 5, or more) strains belonging to V. cholerae O1 classical Hikojima biotype and cholera toxin. In some embodiments, the composition described herein comprises a combination of at least one (e.g., 1, 2, 3, 4, 5, or more) strains belonging to V. cholerae O1 El Tor Hikojima biotype and cholera toxin.

In some embodiments, the composition described herein comprises at least one (e.g., 1, 2, 3, 4, 5, or more) strain belonging to V. cholerae O1 El Tor biotype. In some embodiments, the composition described herein comprises at least one (e.g., 1, 2, 3, 4, 5, or more) strains belonging to V. cholerae O1 classical biotype. In some embodiments, the composition described herein comprises at least one (e.g., 1, 2, 3, 4, 5, or more) strain belonging to V. cholerae O1 El Tor and at least one (e.g., 1, 2, 3, 4, 5, or more) strain belonging to V. cholerae O1 classical biotype. In some embodiments, the composition described herein comprises at least one (e.g., 1, 2, 3, 4, 5, or more) strain belonging to V. cholerae O139. In some embodiments, the composition described herein comprises at least one (e.g., 1, 2, 3, 4, 5, or more) strain belonging to V. cholerae O1 El Tor and/or classical biotype and at least one (e.g., 1, 2, 3, 4, 5, or more) strain belonging to V. cholerae O139.

In some embodiments, the composition described herein comprises at least one (e.g., 1, 2, 3, 4, 5, or more) strain belonging to V. cholerae O1 Inaba El Tor biotype. In some embodiments, the composition described herein comprises at least one (e.g., 1, 2, 3, 4, 5, or more) strain belonging to V. cholerae O1 Ogawa El Tor biotype. In some embodiments, the composition described herein comprises at least one (e.g., 1, 2, 3, 4, 5, or more) strain belonging to V. cholerae O1 Inaba classical biotype. In some embodiments, the composition described herein comprises at least one (e.g., 1, 2, 3, 4, 5, or more) strain belonging to V. cholerae O1 Ogawa classical biotype.

In some embodiments, the composition described herein comprises a combination of at least one (e.g., 1, 2, 3, 4, 5, or more) strain belonging to V. cholerae O1 El Tor biotype and cholera toxin. In some embodiments, the composition described herein comprises at least one (e.g., 1, 2, 3, 4, 5, or more) strains belonging to V. cholerae O1 classical biotype and cholera toxin. In some embodiments, the composition described herein comprises a combination of at least one (e.g., 1, 2, 3, 4, 5, or more) strain belonging to V. cholerae O1 El Tor and at least one (e.g., 1, 2, 3, 4, 5, or more) strain belonging to V. cholerae O1 classical biotype and cholera toxin. In some embodiments, the composition described herein comprises at least one (e.g., 1, 2, 3, 4, 5, or more) strain belonging to V. cholerae O139. In some embodiments, the composition described herein comprises at least one (e.g., 1, 2, 3, 4, 5, or more) strain belonging to V. cholerae O1 El Tor and/or classical biotype and at least one (e.g., 1, 2, 3, 4, 5, or more) strain belonging to V. cholerae O139 and cholera toxin.

In some embodiments, the composition described herein comprises a combination of at least one (e.g., 1, 2, 3, 4, 5, or more) strain belonging to V. cholerae O1 Inaba El Tor biotype and cholera toxin. In some embodiments, the composition described herein comprises a combination of at least one (e.g., 1, 2, 3, 4, 5, or more) strain belonging to V. cholerae O1 Ogawa El Tor biotype and cholera toxin. In some embodiments, the composition described herein comprises a combination of at least one (e.g., 1, 2, 3, 4, 5, or more) strain belonging to V. cholerae O1 Inaba classical biotype and cholera toxin. In some embodiments, the composition described herein comprises a combination of at least one (e.g., 1, 2, 3, 4, 5, or more) strain belonging to V. cholerae O1 Ogawa classical biotype and cholera toxin.

In some embodiments, the composition described herein comprises at least two strains, wherein at least one of the strains belongs to V. cholerae El Tor biotype and at least one of the strains belongs to V. cholerae classical biotype. In some embodiments, the composition described herein comprises at least two strains, wherein at least one of the strains belongs to V. cholerae Ogawa El Tor biotype and at least one of the strains belongs to V. cholerae Ogawa classical biotype. In some embodiments, the composition described herein comprises at least two strains, wherein at least one of the strains belongs to V. cholerae Ogawa El Tor biotype and at least one of the strains belongs to V. cholerae Inaba classical biotype. In some embodiments, the composition described herein comprises at least two strains, wherein at least one of the strains belongs to V. cholerae Ogawa El Tor biotype and at least one of the strains belongs to V. cholerae Inaba El Tor biotype. In some embodiments, the composition described herein comprises at least two strains, wherein at least one of the strains belongs to V. cholerae Ogawa classical biotype and at least one of the strains belongs to V. cholerae Inaba classical biotype. In some embodiments, the composition described herein comprises at least two strains, wherein at least one of the strains belongs to V. cholerae Ogawa classical biotype and at least one of the strains belongs to V. cholerae Inaba El Tor biotype. In some embodiments, the composition described herein comprises at least two strains, wherein at least one of the strains belongs to V. cholerae Inaba classical biotype and at least one of the strains belongs to V. cholerae Inaba El Tor biotype.

In some embodiments, the composition described herein comprises three strains of V. cholerae. In some embodiments, the composition described herein comprises at least three strains, wherein at least one strain belongs to V. cholerae Ogawa El Tor biotype, at least one strain belongs to V. cholerae Ogawa classical biotype, and at least one strain belongs to V. cholerae Inaba classical biotype. In some embodiments, the composition described herein comprises at least three strains, wherein at least one strain belongs to V. cholerae Ogawa El Tor biotype, at least one strain belongs to V. cholerae Inaba classical biotype, and at least one strain belongs to V. cholerae Inaba El Tor biotype. In some embodiments, the composition described herein comprises at least three strains, wherein at least one strain belongs to V. cholerae Ogawa classical biotype, at least one strain belongs to V. cholerae Inaba classical biotype, and at least one strain belongs to V. cholerae Inaba El Tor biotype.

In some embodiments, the composition described herein comprises four strains of V. cholerae. In some embodiments, the composition described herein comprises five strains of V. cholerae. In some embodiments, the composition described herein comprises six or more strains of V. cholerae.

In some embodiments, the composition described herein comprises V. cholerae in the form of whole-cell bacteria. As used herein, the term “whole-cell bacteria” refers to a population of bacteria that are substantially intact bacteria. In some embodiments, the whole-cell bacteria have not been subjected to a process of bacteriolysis or have not been separated into distinct fractions or components. As will be appreciated by one of ordinary skill in the art, whole-cell bacteria may include a portion of bacteria that are not in whole bacterial form, such as a portion of bacteria that have lysed. In some embodiments, the whole-cell bacteria does not contain a substantial amount of lysed bacteria. In some embodiments, at least 50%, 55%, 60%, 65%, 70%, 75%, 80%, 85%, 90%, 95%, or up to 100% of the whole-cell bacteria are in whole bacterial form (e.g., not lysed or fractionated).

Methods for quantifying the amount of whole-cell bacteria in a composition are known in the art and include microscopy methods and assays for detecting bacterial components (e.g., nucleic acid, cytoplasmic components) indicative that the bacteria are not in whole bacterial form.

In some embodiments, the composition described herein contains between 10⁵ and 10¹⁵ cells of each V. cholerae strain per dosage. In some embodiments, the composition contains between 10⁶ and 10¹⁴, between 10⁷ and 10¹³, between 10⁸ and 10¹², between 109 and 10¹¹ cells of each V. cholerae strain per dosage. In some embodiments, the composition contains between 10¹⁰ and 10¹¹ bacterial cells per dosage. In some embodiments, the composition contains approximately 3×10¹⁰ cells of each V. cholerae strain per dosage.

In some embodiments, the composition contains between 10⁵ and 10¹⁵ total V. cholerae cells per dosage. In some embodiments, the composition contains between 10⁵ and 10¹⁵, between 10⁶ and 10¹⁴, between 10⁷ and 10¹³, between 10⁹ and 10¹², between 10¹⁰ and 10¹² total V. cholerae cells per dosage. In some embodiments, the composition contains between 1.0×10¹¹ and 1.5×10¹¹ bacterial cells per dosage. In some embodiments, the composition contains approximately 1.25×10¹¹ total V. cholerae cells per dosage.

In some embodiments, the composition contains between 10⁵ and 10¹⁵ colony-forming units (CFUs) of V. cholerae per dosage. In some embodiments, the composition contains between 10⁵ and 10¹⁵, between 10⁶ and 10¹⁴, between 10⁷ and 10¹³, between 10⁶ and 10⁷, between 10⁸ and 10⁹ total CFUs of V. cholerae per dosage. In some particular embodiments, the composition contains between 10⁸ and 10⁹ bacterial cells per dosage. In more particular embodiment, the composition contains approximately 5×10⁸ total CFUs of V. cholerae per dosage.

In one embodiment, the whole-cell bacteria are live attenuated V. cholerae.

In another embodiment, the whole-cell V. cholerae bacteria are killed or inactivated bacteria. In general, killing or inactivation of whole-cell bacteria means that the bacteria are subjected to a process by which the bacteria is rendered dead or metabolically inactive. A variety of methods of killing or inactivating bacteria are known in the art. For example, the bacteria may be inactivated by chemical inactivation, thermal inactivation, pH inactivation, ionizing radiation inactivation, or UV inactivation. In particular, chemical inactivation or killing involves treatment of bacteria with a chemical agent that include, without limitation, formalin, alcohols, salt, antibiotics, and detergents. The viability or metabolic activity of the bacteria following the process of killing or inactivation may be assessed, for example, by viability staining or plating on growth medium.

In some embodiments, each of the V. cholerae strains of a composition may be inactivated by the same or different method. For example, in some embodiments, the composition may comprise V. cholerae bacteria that have been heat and/or chemically inactivated. In some embodiments, the composition may comprise V. cholerae bacteria that have been heat inactivated. In some embodiments, the composition may comprise at least one V. cholerae strain that has been heat-inactivated. In some embodiments, each of the V. cholerae strains of the composition have been heat inactivated. In some embodiments, the composition may comprise V. cholerae bacteria that have been chemically inactivated. In some embodiments, the composition may comprise V. cholerae bacteria that have been formalin inactivated. In some embodiments, the composition may comprise at least one V. cholerae strain that has been formalin-inactivated. In some embodiment, each of the V. cholerae strains of the composition have been formalin-inactivated.

In some embodiments, the composition may comprise bacteria that have been heat inactivated and bacteria that have been formalin-inactivated. In some embodiments, the composition may comprise bacteria of a V. cholerae strain that has been heat-inactivated and bacteria of the same V. cholerae strain that has been formalin-inactivated. In some embodiments, each of the V. cholerae strains have been inactivated using the same method.

In some embodiments, the composition comprises inactivated bacteria of V. cholerae O1 (subtypes Inaba and/or Ogawa, classical and El Tor biotype,) and V. cholerae O139 strains. Examples of such compositions are cholera vaccines known under the trademarks Shanchol® (Sanofi Oasteur, India), Euvichol® (EUbiologics, Republic of Korea) and mORC-Vax (Vabio Tech, Viet Nam).

The compositions that comprise whole V. cholerae bacteria described herein also comprise cholera toxin associated with the whole V. cholerae cells.

In some embodiments, the whole-cell V. cholerae composition of the invention may further comprise a recombinant cholera toxin (CT) or its B subunit (CTB).

Cholera Toxin Comprising Compositions

Cholera toxin is the main virulence factor produced by the CTXϕ bacteriophage residing in V. cholerae. Cholera toxin is composed of six protein subunits: a single copy of the A subunit and five copies of the B subunit. During infection with V. cholerae, the B subunit ring of the cholera toxin binds to target cells and the entire toxin complex is endocytosed by the cell, leading to release of the cholera toxin A subunit. Subunit B of cholera toxins is not toxic alone. Cholera toxin binds to human cells through interaction between the cholera toxin B subunit with GM1 ganglioside receptors on the cell surface.

Cholera toxin subunit B (CTB) has adjuvant activity for mucosal vaccine; this may be due to the enhanced antigen presentation by various types of antigen-presenting cells, such as macrophages and dendritic cells (Bharati et al. (2011) Indian J. Med. Res. 133: 179-187; Baldauf et al. (2015) Toxins 7: 974-996) In addition to its adjuvant properties, CTB may act as an anti-inflammatory agent by modulating specific signal transduction pathways and may function as an immunomodulatory agent (Royal and Matoba. (2017) Toxins 9(12); Stal et al. (2010) Alimentary Pharmacology and Therapeutics). Oral administration of cholera toxin can upregulate the accumulation of macrophages, natural killer (NK) cells, and the regulatory T cells, as well as IL-10 production, and can downregulate the accumulation of neutrophils (Doulberis et al. (2015) Carcinogenesis 280-290). The immunomodulatory function of CTB may be due to its specific properties, such as the ability of binding to specific GM1 ganglioside receptors present in the gut mucosa, and facilitating antigen uptake and presentation. Previous studies have found that MAPK phosphatase-1 expression can be induced by CTB alone and can subsequently inhibit the activation of Janus kinase and p38, thus leading to a substantial attenuation of TNFα and IL-6 production from macrophages (Chen et al. (2002) J. Immunol. 169:6408-6416).

The present disclosure also includes cholera toxin subunit B variants and cholera toxin subunit A variants. As used herein, the term “cholera toxin subunit B variant” or “cholera toxin subunit A variant” refers to a cholera toxin subunit B or cholera toxin subunit A having at least one amino acid mutation (e.g., insertion, deletion, substitution) relative to the amino acid sequence of a wild type or naturally occurring cholera toxin subunit B or cholera toxin subunit A.

In one embodiment, the composition of the present invention may comprise isolated cholera toxin or its subunit derived from at least one V. cholerae strain expressing the toxin. In another embodiment, the composition described herein may contain the recombinant cholera toxin or its subunit.

In one embodiments, cholera toxin may be obtained from the same V. cholerae strain as the whole bacteria in the composition. In another embodiment, cholera toxin may be obtained from at least one V. cholerae strain different from the strain of the whole bacteria in the composition.

In some embodiments, the composition described herein contains cholera toxin derived from at least one V. cholerae strain that belongs to different O groups. In some embodiments, the composition comprises cholera toxin derived from one or more strains of V. cholerae O1 and one or more strains of V. cholerae belonging to another O group.

In some embodiments, the composition described herein comprises cholera toxin from more than one (e.g., 2, 3, 4, 5, or more) V. cholerae strain. In some embodiments, the composition described herein comprises cholera toxin from at least one (e.g., 1, 2, 3, 4, 5, or more) strain belonging to V. cholerae O1 Inaba classical biotype. In some embodiments, the composition described herein comprises cholera toxin from at least one (e.g., 1, 2, 3, 4, 5, or more) strain belonging to V. cholerae O1 Inaba El Tor biotype. In some embodiments, the composition described herein comprises cholera toxin from at least one (e.g., 1, 2, 3, 4, 5, or more) strain belonging to V. cholerae O1 Ogawa classical biotype. In some embodiments, the composition described herein comprises cholera toxin from at least one (e.g., 1, 2, 3, 4, 5, or more) strain belonging to V. cholerae O1 Inaba El Tor biotype. In some embodiments, the composition described herein comprises cholera toxin from at least one (e.g., 1, 2, 3, 4, 5, or more) strain belonging to V. cholerae O1 Hikojima classical biotype. In some embodiments, the composition described herein comprises cholera toxin from at least one (e.g., 1, 2, 3, 4, 5, or more) strain belonging to V. cholerae O1 Hikojima El Tor biotype.

In some embodiments, the composition described herein comprises cholera toxin from at least two strains, wherein at least one of the strains belongs to V. cholerae El Tor biotype and at least one of the strains belongs to V. cholerae classical biotype. In some embodiments, the composition described herein comprises cholera toxin from at least two strains, wherein at least one of the strains belongs to V. cholerae Ogawa El Tor biotype and at least one of the strains belongs to V. cholerae Ogawa classical biotype. In some embodiments, the composition described herein comprises cholera toxin from at least two strains, wherein at least one of the strains belongs to V. cholerae Ogawa El Tor biotype and at least one of the strains belongs to V. cholerae Inaba classical biotype. In some embodiments, the composition described herein comprises cholera toxin from at least two strains, wherein at least one of the strains belongs to V. cholerae Ogawa El Tor biotype and at least one of the strains belongs to V. cholerae Inaba El Tor biotype. In some embodiments, the composition described herein comprises cholera toxin from at least two strains, wherein at least one of the strains belongs to V. cholerae Ogawa classical biotype and at least one of the strains belongs to V. cholerae Inaba classical biotype. In some embodiments, the composition described herein comprises cholera toxin from at least two strains, wherein at least one of the strains belongs to V. cholerae Ogawa classical biotype and at least one of the strains belongs to V. cholerae Inaba El Tor biotype. In some embodiments, the composition described herein comprises cholera toxin from at least two strains, wherein at least one of the strains belongs to V. cholerae Inaba classical biotype and at least one of the strains belongs to V. cholerae Inaba El Tor biotype.

In some embodiments, the composition described herein comprises cholera toxin from three strains of V. cholerae. In some embodiments, the composition described herein comprises cholera toxin from at least three strains, wherein at least one strain belongs to V. cholerae Ogawa El Tor biotype, at least one strain belongs to V. cholerae Ogawa classical biotype, and at least one strain belongs to V. cholerae Inaba classical biotype. In some embodiments, the composition described herein comprises cholera toxin from at least three strains, wherein at least one strain belongs to V. cholerae Ogawa El Tor biotype, at least one strain belongs to V. cholerae Inaba classical biotype, and at least one strain belongs to V. cholerae Inaba El Tor biotype. In some embodiments, the composition described herein comprises cholera toxin from at least three strains, wherein at least one strain belongs to V. cholerae Ogawa classical biotype, at least one strain belongs to V. cholerae Inaba classical biotype, and at least one strain belongs to V. cholerae Inaba El Tor biotype.

In some embodiments, the composition described herein comprises cholera toxin from four strains of V. cholerae. In some embodiments, the composition described herein comprises cholera toxin from five strains of V. cholerae. In some embodiments, the composition described herein comprises cholera toxin from six or more strains of V. cholerae.

Cholera toxin, including B subunit of cholera toxin, can be obtained by any method known in the art. Methods of obtaining cholera toxin from bacteria are known in the art, for example, utilizing crossflow microfiltration followed by ion exchange chromatography (see, e.g., Jang et al, 2009 J Microbiol Biotechnol. 19(1):10⁸-112), and fractionation onto two successive phosphocellulose columns (see, e.g., Mekalanos et al. 1978. Infect Immun. 20(2): 552-558). In some embodiments, the composition of the present invention may comprise cholera toxin or cholera toxin B subunit that is at least 95.0%, 95.5%, 96.0%, 96.5%, 97.0%, 97.5%, 98.0%, 98.5%, 99.0%, 99.1%, 99.2%, 99.3%, 99.4%, 99.5%, 99.6%, 99.7%, 99.8%, or 99.9% pure.

Alternatively, cholera toxin or a subunit thereof can be produced by recombinant techniques well known in the art, for example by expressing whole toxin or its subunit in a host cell or expression system.

In some embodiments, the composition may comprise pure recombinant cholera toxin or its B subunit (CTB) at the amount from about 0.1 μg to 10 mg, from about 0.1 μs to 5 mg, from about 0.1 μg to 2.5 mg, from about 0.1 μg to 1.5 mg or less per dosage. In some embodiments, the composition may comprise a recombinant CTB at the amount from about 0.75 to 1.5 mg per dosage. In one particular embodiment, the composition comprises about 1 mg of the recombinant CTB per dosage.

In some embodiments, the composition described herein comprises a combination of the whole cells of at least one V. cholerae strain and cholera toxin or CTB obtained from at least one V. cholerae strain.

In a preferred embodiment, the composition described herein comprises a combination the whole cells of at least two V. cholerae strains and cholera toxin or CTB, wherein at least one of the strains belongs to V. cholerae El Tor biotype and at least one of the strains belongs to V. cholerae classical biotype. In some embodiments, the composition described herein comprises a combination of at least two strains and cholera toxin or CTB, wherein at least one of the strains belongs to V. cholerae Ogawa El Tor biotype and at least one of the strains belongs to V. cholerae Ogawa classical biotype. In some embodiments, the composition described herein comprises a combination of at least two strains and cholera toxin or CTB, wherein at least one of the strains belongs to V. cholerae Ogawa El Tor biotype and at least one of the strains belongs to V. cholerae Inaba classical biotype. In some embodiments, the composition described herein comprises a combination of at least two strains and cholera toxin or CTB, wherein at least one of the strains belongs to V. cholerae Ogawa El Tor biotype and at least one of the strains belongs to V. cholerae Inaba El Tor biotype. In some embodiments, the composition described herein comprises a combination of at least two strains and cholera toxin or CTB, wherein at least one of the strains belongs to V. cholerae Ogawa classical biotype and at least one of the strains belongs to V. cholerae Inaba classical biotype. In some embodiments, the composition described herein comprises a combination of at least two strains and cholera toxin or CTB, wherein at least one of the strains belongs to V. cholerae Ogawa classical biotype and at least one of the strains belongs to V. cholerae Inaba El Tor biotype. In some embodiments, the composition described herein comprises a combination of at least two strains and cholera toxin or CTB, wherein at least one of the strains belongs to V. cholerae Inaba classical biotype and at least one of the strains belongs to V. cholerae Inaba El Tor biotype.

In some embodiments, the composition described herein comprises a combination of three strains of V. cholerae and cholera toxin or CTB. In some embodiments, the composition described herein comprises a combination of at least three strains and cholera toxin or CTB, wherein at least one strain belongs to V. cholerae Ogawa El Tor biotype, at least one strain belongs to V. cholerae Ogawa classical biotype, and at least one strain belongs to V. cholerae Inaba classical biotype. In some embodiments, the composition described herein comprises a combination of at least three strains and cholera toxin or CTB, wherein at least one strain belongs to V. cholerae Ogawa El Tor biotype, at least one strain belongs to V. cholerae Inaba classical biotype, and at least one strain belongs to V. cholerae Inaba El Tor biotype. In some embodiments, the composition described herein comprises a combination of at least three strains and cholera toxin or CTB, wherein at least one strain belongs to V. cholerae Ogawa classical biotype, at least one strain belongs to V. cholerae Inaba classical biotype, and at least one strain belongs to V. cholerae Inaba El Tor biotype.

In some embodiments, the composition may comprise a combination of more than three strains, e.g., four, five, six or more strains of V. cholerae and cholera toxin or CTB.

In one particular embodiment, the composition described herein comprises a combination of three strains V. cholerae O1 Inaba, classical biotype; V. cholerae O1 Inaba, El Tor biotype; V. cholerae O1 Ogawa, classical biotype; and cholera toxin or CTB.

In more particular embodiment, the composition comprises the recombinant CTB derived from strains belonging to V. cholerae O1 Inaba classical biotype; V. cholerae O1 Inaba, El Tor biotype; and V. cholerae O1 Ogawa classical biotype.

In more particular embodiment, the composition described herein comprises the inactivated whole-cell bacteria of strains V. cholerae O1 Inaba, classical biotype; V. cholerae O1 Inaba, El Tor biotype; V. cholerae O1 Ogawa, classical biotype and the recombinant CTB. In still more particular embodiment, the composition comprises heat inactivated V. cholerae O1 Inaba, classical biotype; formalin inactivated V. cholerae O1 Inaba, El Tor biotype; heat inactivated V. cholerae O1 Ogawa, classical biotype; formalin inactivated V. cholerae O1 Ogawa, classical biotype; and the recombinant CTB derived from V. cholerae O1 Inaba, classical biotype, strain 213.

In certain embodiment, the composition of the invention comprises all ingredients of the cholera vaccine Dukoral® as described in the patent publication WO2011/034495A1 or EMA summary of product characteristics of Dukoral®.

Briefly, the marketed cholera vaccines contain the active ingredients as listed in Table A and B.

TABLE A
Active ingredients of Dukoral ® vaccine (suspension)

| Active Ingredients | Quantity |
|---|---|
| Vibrio cholerae O1 Inaba, classical | 31.25 × 10⁹ bacteria |
| biotype (heat-inactivated) | |
| Vibrio cholerae O1 Inaba, El Tor | 31.25 × 10⁹ bacteria |
| biotype (formalin-inactivated) | |
| Vibrio cholerae O1 Ogawa, classical | 31.25 × 10⁹ bacteria |
| biotype (heat-inactivated) | |
| Vibrio cholerae O1 Ogawa, classical | 31.25 × 10⁹ bacteria |
| biotype (formalin-inactivated) | |
| Recombinant cholera toxin B subunit | 1 mg |
| (rCTB) | |

TABLE B
Active ingredients of Shanchol ® and Euvichol ® vaccines

| Active Ingredients | Quantity |
|---|---|
| V. cholerae O1 Inaba Cairo 48 | 300 Lipopolysaccharide |
| classical biotype, Heat inactivated | ELISA Units (L.E.U.) |
| V. cholerae O1 Inaba Phil 6973 El | 600 L.E.U |
| Tor biotype, Formalin inactivated | |
| V. cholerae O1 Ogawa Cairo 50 | 300 L.E.U |
| classical biotype, Formalin | |
| V. cholerae O1 Ogawa Cairo 50 | 300 L.E.U |
| classical biotype, Heat inactivated | |
| V. cholerae O139 4260B, Formalin | 600 L.E.U |
| inactivated | |

Pharmaceutical Compositions

According to the present disclosure, the compositions comprising at least one virus, or a bacterial strain, and/or a bacterial protein such as a toxin are prepared as pharmaceutical compositions. The term “pharmaceutical composition” as used herein means a product that results from the mixing or combining of more than one active ingredient to permit the biological activity of the active ingredients and which contains no components which are toxic to the subject to which the composition would be administered. The term “pharmaceutical composition” also includes fixed and non-fixed combinations of the active ingredients. The term “fixed combination” means that the active ingredients, e.g. at least one virus or bacterial strain, or a protein, or a toxin or a combination thereof, and a co-agent (e.g. adjuvant), are both administered to a patient simultaneously in the form of a single entity or dosage. The term “non-fixed combination” means that the active ingredients, e.g. at least one virus or bacterial strain, or a protein, or a toxin or a combination thereof, and a co-agent (e.g. adjuvant), are both administered to a patient as separate entities either simultaneously, concurrently or sequentially with no specific time limits.

In some embodiments, the pharmaceutical compositions of the present invention can be formulated readily by combining the compounds with pharmaceutically acceptable carriers and/or excipients, also known in the art as stabilizers, preservatives, buffers, solubilizers, surfactants, osmolytes, food (flavor) additives. Carriers enable the active compounds to be formulated as a powder, granules, tablets, pills, dragees, capsules, and alike. The suitable excipients are, in particular, fillers such as sugars (carbohydrates), including monosaccharides (e.g. galactose, mannose, sorbose, etc.), disaccharides (e.g., sucrose, trehalose, lactose, etc.) and polysaccharides such as dextran, cellulose, maize starch, wheat starch, rice starch, potato starch, methyl cellulose, hydroxypropylmethyl-cellulose, sodium carboxymethylcellulose, and cyclodextrin; human and bovine serum albumin, egg albumin, gelatin, immunoglobulin, skim milk powder, casein, soya protein, wheat protein and any protein hydrolysates; amino acids (e.g., leucine, lysine, alanine, arginine, histidine, glutamate, etc.); methylamines such as betaine; polyols such as sugar alcohol (e.g. glycerin, glycerol, sorbitol, arabitol, erythitol, mannitol, etc.); synthetic polymers such as propylene glycol, polyethylene glycol, polyvinylpyrrolidone, pluronics, and others (see e.g. Handbook of Pharmaceutical Excipients, 4th Edition, Rowe et al., Eds., Pharmaceutical Press (2003)).

In some embodiments, the compositions may further comprise sufficient amounts of protecting agents, which preserve structural or functional features of the biological material and viability of live bacteria or viruses. If desired, disintegrating agents, such as agar, alginic acid or sodium alginate may be added.

In some embodiments, the pharmaceutical compositions of the present invention may comprise a buffer, such as phosphate (e.g. sodium phosphate, potassium phosphate, or a mixture of the two; 0.1% to 2% w/w), histidine (0.5% w/w or 2.5 to 50 mM), citrate, acetate, succinate or lactate buffer.

In some embodiments, the pharmaceutical compositions of the present invention can range in pH from pH 5.5 to pH 8.5 at room temperature. In certain embodiments, the pH range is from pH 6.0 to pH 8.0. In more certain embodiments, the pH range is from pH 6.5 to pH 7.5. In one particular embodiments, the pH range is from pH 6.8 to pH 7.2. In a preferred embodiment, the pH is about pH 7.0.

In certain embodiments, the pharmaceutical compositions of the present invention may further comprise one or more divalent cation(s) or a salt of a cation. In certain embodiments, the cation is calcium (Ca′). In other embodiments, the cation is magnesium (Mg′). In still other embodiments, the cation is zinc (Zn′). In yet other embodiment, the cation is a mixture of Ca′, Mg′ and/or Zn′. It has been shown, that divalent cations improve stability of several viral vaccines. For instance, a combination of Zn′ and Ca′ improved the storage stability of a spray dried live attenuated measles vaccine by one log TCID₅₀ when stored for one week at 37° C. (see Ohtake et al., 2010). The exact nature of this cation stabilizing is not clearly understood but it was hypothesized that divalent cations interact with the membrane lipids and proteins and thereby preserve integrity of viral structure during processing. The pharmaceutical composition may be formulated into pharmaceutically acceptable dosage forms by conventional methods known to those of skill in the art.

In a particular embodiment, the pharmaceutical composition of the present invention is a vaccine capable to elicit an immune response in a subject upon administration. The immune response may include humoral immune response and/or cellular immune response. The vaccine composition described herein may activate B and/or T cells and therefore provide protective immunity to a subject.

In some embodiments, the vaccine composition of the present invention can further include at least one immunologically active adjuvant selected from the group but not limited to aluminium salt (alum), monophosphoryl lipid A, QS-21, ISCOMS, saponins, polycationic polymers such as polyarginine or a peptide containing at least two LysLeuLys motifs, especially KLKLLLLLKLK, immunostimulatory oligodeoxynucleotide (ODN) containing non-methylated cytosine-guanine dinucleotides (CpG) in a defined base context (e.g. as described in WO 96/02555) or ODNs based on inosine and cytidine (e.g. as described in WO 01/93903), or deoxynucleic acid containing deoxy-inosine and/or deoxyuridine residues (as described in WO 01/93905 and WO 02/095027), especially Oligo(dIdC)13 (as described in WO 01/93903 and WO 01/93905), or combinations thereof such as IC31® (Valneva SE).

In a preferred embodiment, the pharmaceutical composition of the present invention is a cholera vaccine comprising the whole-cell bacteria of V. cholerae and B subunit of cholera toxin (CTB) as described above.

Dry Formulations

According to the present invention, the pharmaceutical compositions including vaccines are prepared as dry formulations, which maintain their biological activities and/or efficacies upon drying. Usually, dry formulations are more stable at non-refrigerated temperatures as compared to their liquid counterparts. Stability of dry compositions that comprise biological materials increase partially due to decreased mobility of biological ingredients such as proteins or lipids (e.g. LPS) and partially due to prevention of degradation pathways facilitated by water. Additionally, stability of dry pharmaceutical compositions is improved in the presence of stabilizers.

In one embodiment, the dry formulation of the present invention comprises a sufficient amount of a stabilizing agent. Examples of stabilizing agents include, but are not limited to, carbohydrates including monosaccharides (e.g. galactose, mannose, sorbose), disaccharides (e.g., sucrose, trehalose, lactose) and polysaccharides (e.g. dextran, maltodextrin, cellulose), polyols such as sugar alcohol (e.g. glycerin, glycerol, sorbitol, arabitol, erythitol, mannitol), amino acids (e.g., leucine, lysine, alanine, arginine, histidine, glutamate), etc. In a particular embodiment, the stabilizer is a sugar. In more particular embodiment, the stabilizer is selected from the group consisting of sucrose, tregalose, raffinose, lactose, maltose, mannitol, sorbitol, maltodextrin, arginine, histidine, glycine, or variable combinations thereof. In a preferred embodiment, the stabilizer is sucrose or maltodextrin.

In some embodiments, the dry formulation of the invention comprises, in percent by weight of total dry content, about 10% to 90% (w/w) of a stabilizer. In particular, the amount of stabilizer can be about 10%, 20%, 30%, 40%, 50%, 60%, 70%, 80% and 90% (w/w) of total composition content. In a preferred embodiment, the dry formulation comprises between 50% and 90% (w/w) of the stabilizer.

In other embodiments, the stabilizer is a combination of two or more stabilizers. In a particular embodiment, the stabilizer is a combination of two sugar stabilizers used in a ratio 1:1, 1:2, 1:3, 1:4, 1:5, 1:6, 1:7, 1:8, 1:9, 1:10; 1:20, 1:50, etc.

In a particular embodiment, the dry formulation described herein comprises the combination of sucrose and maltodextrin as a stabilizing agent. In one particular embodiment, sucrose and maltodextrin are present in the formulation in the ratio 1:4 respectively. In still one particular embodiment, sucrose and maltodextrin are present in the ratio 1:9 respectively.

In one particular embodiment, for example, the dry formulation described herein may comprise, in percent of total dry content, about 1% to 10% (w/w) of a bioactive material, about 0 to 20% (w/w) of sucrose and about 70% to 90% (w/w) of maltodextrin. More specifically, the dry formulation may comprise, in percent of total dry content, about 1-10% (w/w) of a bioactive material, 20% (w/w) of sucrose and 70% (w/w); or about from 1-10% of the bioactive material, 10% of sucrose and 80% of maltodextrin; or about 1-10% (w/w) of the bioactive material, and 90% of maltodextrin.

In another particular embodiment, for example, the dry formulation described herein may comprise sucrose in a concentration ranging from about 0 to 50 mg/ml, more particularly about 0 to 40 mg/ml, specifically about 0, 16 or 32 mg/mL.

In another particular embodiment, the dry formulation described may comprise maltodextrin in a concentration ranging from about 120 to 170 mg/mL, more particularly about 130 to 165 mg/mL, specifically about 130 mg/mL, about 147 mg/mL, or about 164 mg/mL.

In a preferred embodiment, the dry formulation is a dried cholera vaccine described herein that comprises at least one V. cholerae strain with or without cholera toxin (CTB), and further comprises at least one stabilizer. In more particular embodiment, the dry cholera vaccine formulation comprises at least one V. cholerae strain or a combination of at least one V. cholerae strain and cholera toxin (CTB) and a sugar stabilizer. In even more particular embodiment, the dry cholera vaccine formulation comprises at least one V. cholerae strain or a combination of at least one V. cholerae strain and cholera toxin (CTB) and the sugar stabilizer(s) such as sucrose and/or maltodextrin.

In still more preferred embodiment of the present invention, the dry cholera vaccine formulation comprises all ingredients (including or not including rCTB) of the cholera vaccine known under the trade name Dukoral® (see Table A) admixed with the sugar stabilizer.

In one particular embodiment, the dry cholera vaccine (one dosage unit) comprises in total between about 1.0×10″ and 1.5×10″, preferably about 1.25×10″ whole-cell bacteria of the following strains:
Vibrio cholerae O1 Inaba, classical biotype (heat inactivated),
Vibrio cholerae O1 Inaba, El Tor biotype (formalin inactivated),
Vibrio cholerae O1 Ogawa, classical biotype (heat inactivated),
Vibrio cholerae O1 Ogawa, classical biotype (formalin inactivated), excipients: sodium dihydrogen phosphate dihydrate (2.0 mg), disodium hydrogen phosphate dihydrate (9.4 mg), sodium chloride (26 mg), and further comprising the sugar stabilizer such as sucrose and/or maltodextrin.

In yet one particular embodiment, the dry cholera vaccine (one dosage unit) comprises in total between about 1.0×10¹¹ and 1.5×10¹¹, preferably about 1.25×10¹¹ whole-cell bacteria of the following strains:
Vibrio cholerae O1 Inaba, classical biotype (heat inactivated),
Vibrio cholerae O1 Inaba, El Tor biotype (formalin inactivated),
Vibrio cholerae O1 Ogawa, classical biotype (heat inactivated),
Vibrio cholerae O1 Ogawa, classical biotype (formalin inactivated), a recombinant cholera toxin B subunit (rCTB) (0.75-1.5 mg, preferably 1.0 mg), excipients: sodium dihydrogen phosphate monohydrate (2.0 mg), disodium hydrogen phosphate dihydrate (9.4 mg), sodium chloride (26 mg), and further comprising the sugar stabilizer such as sucrose and/or maltodextrin,
Usually, the total amount of whole-cell bacteria is calculated before bacteria inactivation and vaccine drying.

Preferably, the ratio of the dried cholera vaccine and the sugar stabilizer (sucrose and/or maltodextin) is, in total dry content, 1:10 (w/w). More preferably, sucrose and maltodextrin in said formulation may be used in a ratio 0:1, 1:9 or 1:4, respectively.

In one preferred embodiment, the dry cholera vaccine formulation comprises, in percent of total dry content, about 10% (w/w) of Dukoral® and about 90% (w/w) of the stabilizer comprising sucrose and/or maltodextrin. In another preferred embodiment, the dry cholera vaccine formulation comprises, in percent of total dry content, about 10% (w/w) of Dukoral® and about 90% (w/w) of the stabilizer comprising sucrose and/or maltodextrin.

Even more preferably, the dry cholera vaccine formulation comprises about 16.4 mg of dry Dukoral® and 164 mg of the stabilizer. In one preferred embodiment, the dry cholera vaccine formulation comprises about 16.4 mg/mL of the dry Dukoral® and about 164 mg/mL of maltodextrin. In another preferred embodiment, the dry cholera vaccine formulation comprises about 16.4 mg/mL of the dry Dukoral®, about 148 mg/mL of maltodextrin and about 16 mg/mL of sucrose. In yet another preferred embodiment, the dry cholera vaccine formulation comprises about 16.4 mg/mL of the dry Dukoral®, about 132 mg/ml of maltodextrin and about 32 mg/mL of sucrose. More specifically, in one example, the dry cholera vaccine formulation comprises exactly 16.4 mg/mL of the dry Dukoral®, 147.6 mg/mL of maltodextrin and 16.4 mg/mL of sucrose. In another example, the dry cholera vaccine formulation comprises exactly 16.4 mg/mL of the dry Dukoral®, 131.2 mg/ml of maltodextrin and 32.8 mg/mL of sucrose.

The dry pharmaceutical compositions of the invention are characterized by low moisture content (or water content). Residual moisture content is one of the critical factors that impact physical or chemical stability and potency of the dry composition during long-term storage. Usually, the recommended residual moisture content for stable lyophilized materials is in the range of 0.5%-3% (w/w). For instance, the residual moisture content of lyophilized Influenza antigen with 1% sucrose is between 0.5% w/w (by colometric Karl Fischer method) and 0.81% w/w (by TGA) (see https://www.americanpharmaceuticalreview.com/Featured-Articles/116129-Analytical-Options-for-the-Measurement-of-Residual-Moisture-Content-in-Lyophized-Biological -Materials/).

In one embodiment, the residual water content of the dry composition of the invention is equal to or less than 3%. In yet one embodiment, the residual water content of the dry composition of the invention is between 3% and 1%. In a preferred embodiment, the residual water content of the dry composition of the invention is between 3% and 2%.

Another critical factor that impacts stability of the compositions is water activity. Water activity (a_{w}) is the partial vapor pressure of water in a substance divided by the standard state partial vapor pressure of water:

a_{w}=p/p*,

where p is the partial vapor pressure of water in the solution, and p* is the partial vapor pressure of pure water at the same temperature.

Alternate definition of water activity (a_{w}):

a_{w}=l_{w}x_{w}

where l_{w} is the activity coefficient of water and x_{w} is the mole fraction of water in the aqueous fraction.

TABLE C
Examples of a_{w} values (* - a_{w} of growth of the bacterium)

| Substance/microorganism | a_{w} | Reference |
|---|---|---|
| Distilled water | 1.00 | (1) |
| Raw meat | 0.99 | (1) |
| Milk | 0.97 | (1) |
| Dried fruit | 0.60 | (1) |
| Peanut butter | 0.35 | (2) |
| Clostridium botulinum E* | 0.97 | (3) |
| Escherichia coli* | 0.95 | (3) |
| Vibrio cholerae* | 0.95 | (3) |
| Bacillus subtilis* | 0.91 | (3) |
| Staphylococcus aureus* | 0.86 | (3) |
| No microbial proliferation* | <0.60 | (3) |

(1) Marianski, Stanley; Marianski, Adam (2008). The Art of Making Fermented Sausages. Denver, Colorado: Outskirts Press.
(2) He, Y.; Li, Y.; Salazar, J. K.; Yang, J.; Tortorello, M. L.; Zhang, W. (2013). “Increased Water Activity Reduces the Thermal Resistance of Salmonella enterica in Peanut Butter”. Applied and Environmental Microbiology. 79 (15): 4763-4767.
(3) Barbosa-Canovas, G.; Fontana, A.; Schmidt, S.; T. P. (2007). “Appendix D: Minimum Water Activity Limits for Growth of Microorganisms”. Water Activity in Foods: Fundamentals and Applications.

Water activity values are usually obtained by either a resistive electrolytic, a capacitance or a dew point hygrometer. In a particular embodiment, the water activity (a_{w}) in the powder samples are measurement using a Water Activity Meter (AquaLab 4TE) and characterized by dew point.

Water activity is related to water content in a non-linear relationship known as a moisture sorption isotherm curve. The isotherm is substance- and temperature-specific. The isotherm can be used to help predict product stability over time in different storage conditions.

According to the present invention, the dry composition has a water activity equal to or less than about 0.15, preferably between 0.15 and 0.02, particularly about 0.14, 0.13, 0.12, 0.11, 0.1, 0.09, 0.08, 0.07, 0.06, 0.05, 0.04, or 0.03. In one preferred embodiment, the dry composition has a water activity equal to or less than about 0.1. In another preferred embodiment, the dry composition has a water activity about 0.03. In a preferred embodiment, the dry composition that has a water activity equal to or less than 0.15 is a vaccine. In more preferred embodiment, the dry composition that has a water activity equal to or less than 0.15 is a cholera vaccine. In one more preferred embodiment, the dry composition that has a water activity equal to or less than 0.1 is a cholera vaccine.

Examples of the cholera vaccine compositions are disclosed above. One particular example of the cholera vaccine composition is Dukoral®. Preferably, the dry Dukoral® formulation has a water activity of equal to less than 0.15. More preferably, the dry Dukoral® formulation has a water activity of equal to less than 0.1. Even more preferably, the dry Dukoral® formulation has a water activity between 0.1 and 0.02. In one preferred embodiment, the dry Dukoral® formulation has a water activity about 0.03.

In one embodiment, the dry pharmaceutical compositions or formulations of the invention including dry cholera vaccines that has a water activity equal to or less than 0.15 are more stable than the corresponding compositions or formulations that has a water activity more than 0.15.

In a certain embodiment, the dry pharmaceutical compositions or formulations of the invention are more stable under certain storage conditions than their liquid counterparts. A “stable” composition or formulation is one in which the biologically active material essentially retains its physical stability and/or chemical stability and/or biological activity upon storage. Various analytical techniques for measuring stability are available in the art and are reviewed, e.g., in Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993). Among storage conditions, temperature is the most critical. Generally, the recommended storage temperature for the vaccines, including dry vaccines, is between −20° C. and +8° C., usually between +2° C. and +8° C. In particular, the marketed cholera vaccines, including Dukoral®, can be stored at refrigerated temperature for more than one year.

The dry composition or formulation of the invention, including vaccine, is stable at elevated temperatures, such as between about 20° C. and 40° C., particularly between about 25° C. and 35° C., especially at about 25° C. or 30° C. for at least one year or even longer. Particularly, the dry composition or formulation of the invention, including vaccine, is stable at a temperature between 20° C. and 40° C. for at least one year, two years, three years, four years, or five years. Preferably, the dry compositions or formulations of the present invention, including vaccines, are stable at about 25° C. or 30° C. for at least two years.

Due to elevated thermostability, the dry composition or formulation which has a water activity equal to or less than 0.15 has prolonged storage life at a temperature between about 20° C. and 40° C., particularly between about 25° C. and 35° C., especially at about 25° C. or 30° C., as compared to a composition that has a water activity of more than 0.15.

More specifically, the dry composition or formulation of the invention, including vaccine, can be stored at a temperature between about 20° C. and 40° C., particularly between about 25° C. and 35° C., especially at about 25° C. or 30° C., for at least one year. Preferably, the dry composition or formulation of the invention, including vaccine, can be stored at a temperature between about 20° C. and 40° C., particularly between about 20° C. and 30° C., especially at about 25° C. or 30° C., for at least two years. More preferably, the dry composition or formulation of the invention, including vaccine, can be stored at a temperature between about 20° C. and 40° C., particularly between about 25° C. and 35° C., especially at about 25° C. or 30° C., for at least three years.

Importantly, potency of the dry composition or formulation of the invention does not change significantly upon storage under the elevated temperature, particularly at a temperature between about 20° C. and 40° C., more particularly between about 25° C. and 35° C., especially at about 25° C. or 30° C., for at least one year, preferably for two or more years. In one particular embodiment, potency of the dry composition that has a water activity equal to or less than 0.15 does not deviate more than +/−50% upon storage for at least one year at a temperature between about 20° C. and 40° C., as compared to the same composition having a water activity more than 0.15. In a preferred embodiment, potency of the dry composition that has a water activity equal to or less than 0.15 does not deviate more than +/−30% upon storage for at least one year at a temperature between about 20° C. and 40° C., as compared to the same composition having a water activity more than 0.15.

In one particular embodiment, the stable dry vaccine of the invention is a cholera vaccine comprising at least one V. cholera strain, and further comprising at least one stabilizer. In a preferred embodiment, the stable dry cholera vaccine has a water activity equal to or less than 0.15. In more preferred embodiment, the stable dry cholera vaccine has a water activity equal to or less than 0.1.

In a preferred embodiment, the dry cholera vaccine that has a water activity equal to or less than 0.15 has prolonged storage life as compared to the counterpart composition that has a water activity of more than 0.15 under the same storage conditions. In a preferred embodiment, the dry cholera vaccine that has a water activity equal to or less than 0.15 has prolonged storage life when stored at a temperature between 20° C. and 40° C., or between 25° C. and 35° C., preferably at 25° C. or 30° C. as compared to a composition that has a water activity of more than 0.15. In a preferred embodiment, the dry cholera vaccine that has a water activity equal to or less than 0.15 has storage life at least one year, preferably two or more years, at a temperature between 25° C. and 35° C. In still one embodiment, the dry composition of the invention including dry vaccine that has a water activity equal to or less than 0.15 has storage life at least one year, preferably two or more years, at a temperature about 25° C. or 30° C. as compared to a composition that has a water activity of more than 0.15.

In additional preferred embodiment, the dry cholera vaccine that has a water activity equal to or less than 0.15 retains its potency significantly unchanged upon storage for at least one year, preferably two or more years, at a temperature between 20° C. and 40° C. In a preferred embodiment, potency of the dry cholera vaccine that has a water activity equal to or less than 0.15 does not deviated more than +/−50% upon storage for at least one year, preferably two or more years, at a temperature between about 20° C. to 40° C. In one more preferred embodiment, potency of the dry cholera vaccine that has a water activity equal to or less than 0.15 does not deviated more than +/−50% upon storage for at least one year, preferably two or more years, at a temperature between about 25° C. to 35° C. In still one preferred embodiment, potency of the dry cholera vaccine that has a water activity equal to or less than 0.15 does not deviated more than +/−50% upon storage for at least one year, preferably two or more years, at about 25° C. or 30° C.

In another preferred embodiment, potency of the dry cholera vaccine that has a water activity equal to or less than 0.15 vaccine does not deviated more than +/−30% upon storage for at least one year, preferably two or more years, at a temperature between about 20° C. to 40° C. In still another preferred embodiment, potency of the dry cholera vaccine that has a water activity equal to or less than 0.15 does not deviated more than +/−30% upon storage for at least one year, preferably two or more years, at a temperature between about 25° C. to 35° C. In still another preferred embodiment, potency of the dry cholera vaccine that has a water activity equal to or less than 0.15 does not deviated more than +/−30% upon storage for at least one year, preferably two or more years, at temperature about 25° C. or 30° C. In a particularly preferred embodiment, potency of the dry cholera vaccine that has a water activity equal to or less than 0.15 does not deviate more than +/−30% upon storage for at least two years at a temperature about 25° C.

In a certain embodiment, potency of the dry cholera vaccine that has a water activity equal to or less than 0.15 does not decrease more than potency of the corresponding liquid cholera vaccine formulation or formulation that has a water activity more than 0.15 upon storage at the same storage conditions, particularly upon storage at least one year at a temperature between about 20° C. to 40° C., preferably at about 25° C. or 30° C.

In one particular embodiment, stability of the dry cholera vaccine composition is evaluated based on stability of V. cholerae bacteria assessed by an LPS assay. In this assay the presence of LPS antigen on the surface of V. cholerae is measured by ELISA in the reconstituted sample. In another embodiment, stability of the dry cholera vaccine composition comprising the recombinant CTB is evaluated by measuring stability of the CTB antigen as described by Mancini et al. (Mancini G, Carbonara AO, and Heremans J F. 1965. Immunochemical quantitation of antigens by single radial immunodiffusion. Immunochemistry 2: 235-254). In yet one embodiment, stability of the dry vaccine composition is evaluated by the bacterial count at OD₆₀₀ in the reconstituted sample.

Methods of Preparing Stable Dry Formulations

Dry pharmaceutical compositions of the present invention, including vaccines, can be obtained or are obtainable by drying of known liquid formulations. Dry pharmaceutical compositions can be processed according to the methods well known in the art (see e.g., Remington: The Science and Practice of Pharmacy, Mack Publishing Co. 20th ed. 2000; and Ingredients of Vaccines—Fact Sheet from the Centers for Disease Control and Prevention, e.g., adjuvants, enhancers, preservatives, and stabilizers). Such methods, in particular, include freeze drying, spray drying and modifications thereof.

Freeze drying or lyophilization is well known and widely used for preparing dry formulations of protein and viral/bacterial compositions. By this method many vaccines such as lyophilized Hiberix® (GSK), Rotavix® (GSK), Varivax® (Merck), Imovax® (Sanofi Pasteur), YFVax® (Sanofi Pasteur), Menomune (Sanofi Pasteur), Varivax® (Merck), MMR II (Merck), JE-Vax (Osaka) have been prepared. The lyophilization process involves freezing of a liquid solution followed by controlled removal of water by sublimation of ice (so called primary drying) and thereafter by desorption of remaining water at low pressure and higher temperature (so called secondary drying). This results in a dried cake in the final container and requires reconstitution before administration. One drawback of using lyophilization for preparing dry compositions comprising biologically active materials, such as bacteria or virus, is partial damage of biomaterials during drying (see e.g. Ohtake et al., 2010). For protection of biomaterials against damage during lyophilization and increasing their stability during storage, usually stabilizers are added before drying. Additionally, considering that most vaccines are heat sensitive, it is crucial to use optimized process parameters such as optimal lowest and highest temperatures (usually −65° C. and +20° C.), flow rate, pressure, gas, moisture, etc. to minimize biomaterial damage.

Spray drying, an alternative to freeze-drying, is a continuous one-step process for producing bulk powder vaccines well known in the art (see e.g. Kanojia et al., 2017; WO2016009400).

Briefly, the process converts a liquid feed (liquid containing vaccine and stabilizers) into fine dispersible particles (aerosol) then dried in heated gaseous medium. The drying gas is at a pressure that allows it to flow at the range of 25 m³/h to 55 m³/h with inlet temperature ranging from 0° C. to +200° C., preferably +180° C., and outlet temperature ranging from +35° C. to +100° C., preferably +90° C. Flow rate of the feed suspension is at the range of 0.3 mL/min to 10.0 mL/in, preferably from 1 mL/min to 5 mL/min, more preferably about 5 mL/min. Spray drying process results in a fine powder, which can be easily formulated into pharmaceutically acceptable dosage forms or delivered without reconstitution to, for example, mucosal routs of administration.

Formulation of a dosage form typically involves combining an active ingredient and one or more excipients; the resultant dosage form determines the route of administration and the medical efficacy (for review see e.g. Jahan et al. 2019).

Generally, dry pharmaceutical composition, including solid vaccines, may be produced in different dosage forms, such as various types of tablets, capsules, granules, sachets, reconstitutable powders, powders, dry-powder inhalers, chewables, injectors, microneedles, films and others. In a preferred embodiment of the invention, the dry vaccine composition is produced as a powder or capsules.

Methods of Administration and Use

The dry compositions of invention including dry vaccines may be formulated in dosage form suitable for parenteral administration by injection. As used herein, “parenteral” administration includes, without limitation, subcutaneous, intracutaneous, transdermal, intravenous, intramuscular, intraarticular, intrathecal, intravaginal or by infusion. Formulations for injection have to be aqueous solutions or suspensions of active ingredients. The dry compositions require reconstitution with a suitable vehicle immediately before use. Optionally, the dry compositions may be reconstituted in sterile water, saline or buffers to form solution or suspension for injection or oral delivery.

Alternatively, the dry compositions can be injected as solids, e.g. when the solid is a powder and the injector is a needleless powder injector, such as PowderJect®, or as coated or dissolving microneedles (see e.g. Jahan et al., 2019).

Preferably, the compositions of the present invention including dry vaccines can be administered to the subject via oral, intranasal, buccal, sublingual or pulmonary (by inhalation) route. The oral route is always one of the most desired. For oral administration, the dry compositions can be formulated in form of powder, granules, tablets, or capsules. Mucosal delivery of the vaccine has an advantage associated with inducing mucosal immunity at the port of entry of the pathogen, potentially providing the first line of protection as compared to parenteral vaccine delivery.

According to the present invention, the dry vaccine formulation comprising inactivated whole-cell V. cholerae alone or in combination with the recombinant cholera toxin (CTB) can be administered to the subjects orally in dry form as a capsule or as a powder reconstituted in a buffer, e.g. sodium carbonate buffer, immediately before use. Particularly, a buffer is sodium hydrogen carbonate buffer, which contains approximately 1 g, preferably 1.1 g sodium per dosage. In a preferred embodiment, the reconstitution buffer comprises sodium hydrogen carbonate (3600 mg), sodium carbonate anhydrous (400 mg), saccharin sodium (30 mg), sodium citrate (6 mg) and citric acid (1450 mg) per dose (3 ml). Optionally, for delivery via digestive route the reconstitution buffer may comprise a flavor.

Any of the compositions described herein including dry vaccine formulations may be administered to a subject once, twice, three times or more, e.g. as a triple or quadruple dose or as a booster dose one month, two months, three months or more after the first dose. In a particular embodiment, the dry compositions comprising V. cholerae including dry cholera vaccine described herein may be administered to the subject once, twice or more. In more particular embodiment, the dry cholera vaccine comprising V. cholerae and optionally cholera toxin may be administered to a subject more than once (e.g., as multiple doses), preferably at least twice. In one particular embodiment, the dry cholera vaccine described herein may be administered to a subject three times

In some embodiments, the more than one administration of the composition described herein are delivered sequentially to the subject. In some embodiments, a subsequent administration of the composition described herein is administered at least 1 week, 2 weeks, 3 weeks, 4 weeks, 5 weeks, 6 weeks, 7 weeks, 8 weeks, 9 weeks, 10 weeks, 11 weeks, 12 weeks, or longer after the first administration. In one particular embodiments, a subsequent administration of the composition comprising V. cholerae including the dry cholera vaccine is administered at least 1, 2, 3, 4, 5, 6 weeks but not more than 60 days after the first administration. Determining whether a subject is in need of one or more additional administrations of the composition described herein will be evident to one of ordinary skill in the art.

In some embodiments, the dry compositions described herein may be used for infection treatment. As used herein, the terms “treatment”, “treat” and “treating,” include prevention, cure, amelioration, reducing or delay the onset of the symptoms, complications, pathologies or biochemical indicia of a disease. Treatment may be prophylactic (to prevent or delay the onset of the disease, or to prevent or reduce the manifestation of clinical or subclinical symptoms thereof) or therapeutic alleviation of symptoms after the manifestation of the disease.

In some embodiments, the dry composition described herein may be used for the treatment of a viral infection in the subjects. The viral infection may be caused by of Adenovirus, Chikungunia virus, Coronavirus, SARS-CoV2, Cytomegalovirus, Dengue virus, Epstain-Barr virus, Ebola virus, Enterovirus, Influenza virus, Japanese Encephalitis virus, Hepatitis A virus, Hepatitis B virus, Hepatitis C virus, human Immunodeficiency virus, human papilloma virus, Herpes Simplex virus, Herpes Zoster virus, human Methapneumovirus, human rhinovirus, Measles virus, Mumps virus, paramyxovirus, Parvovirus B19, polyovirus, human parainfluenza virus, Rabies virus, Respiratory Syncytial virus, Rubella virus, Rotavirus, Smallpox virus, tick borne encephalitis virus, Varicella-zoster virus, Vaccinia virus, West Nile virus, Yellow Fever virus, or Zika virus.

In other embodiments, the dry composition described herein may be used for the treatment of a bacterial infection in the subjects. The bacterial infection may be caused by any bacteria of the group consisting of Bacillus anthracis, Bordetella bronchiceptica, Bordetella pertussis, Borrelia burgdorferi, Brucella abortis, Brucella species, Candida albicans, Chlamydia pneumonia, Chlamidia trachomatis, Chlamidia psittaci, Clostridium difficile, Clostridium perfringens, Clostridium botulinum, Clostridium tetani, Corynebacterium diphtheria, Enterococcus faecalis, Enterobacter species, Escherichia coli, Helicobacter pylori, Haemophilus influenza, Klebsiella pneumohiae, Legionella pneumophila, Leishmania species, Listeria monocytogenes, Mycobacterium leprae, Mycobacterium tuberculosis, Mycoplasma species, Niesseria meningitides, Niesseria gonorrhoeae, Pseudomonas aeruginosa, Salmonella thyphimurium, Shigella dysentheriae, Shigella shinga, Staphylococcus aureus, Staphylococcus epidermidis, Streptococcus pneumoniae, Streptococcus agalactiae, Streptococcus pyogenes, Vibrio cholerae, Vibrio parahaemolyticus, Yersinia entercolitica, and Yersinia pestis.

In a particular embodiment, the dry cholera vaccine described herein may be used for the treatment of V. cholerae infection in the subject. The treatment includes the administration of the composition comprising at least one V. cholerae strain, the composition comprising the combination of at least one V. cholerae strain and cholera toxin or cholera toxin B subunit (CTB) to the subject in order to prevent, cure, ameliorate, reduce, or delay the onset of the symptoms, complications, pathologies or biochemical indicia of cholera disease.

Any of the compositions described herein may be administered to a subject of need in a therapeutically effective amount. As used herein, a “therapeutically effective amount” or an “effective amount” of composition is any amount that results in a desired response or outcome in a subject, such as those described herein, including but not limited to preventing or treating an infection.

In particular, the dry composition comprising V. cholerae bacteria with or without cholera toxin (CTB) described herein, e.g. the dry cholera vaccine, may be administered to a subject of need in a therapeutically effective amount.

In one embodiments, the dosage of the dry composition comprising V. cholerae including the dry cholera vaccine refers to the amount of V. cholerae bacteria that is administered to the subject within the composition.

In some embodiments, the composition described herein may contain between 10⁵ and 10¹⁵ cells of total V. cholerae bacteria per dosage. In some embodiments, the compositions contain between 10⁵ and 10¹⁵, between 10⁶ and 10¹⁴, between 10⁷ and 10¹³, between 10⁸ and 10¹², between 10⁹ and 10¹¹, or about 10¹¹ cells of total V. cholerae bacteria per dosage. In one particular embodiments, the composition may contain approximately 10¹¹ V. cholerae cells per dosage. In yet one particular embodiment, the composition contains approximately 1.25×10¹¹ total V. cholerae cells per dosage. In yet one particular embodiment, the composition contains approximately 3×10¹⁰ cells of each V. cholerae strain per dosage.

In some embodiments, the composition of the invention may contain between 10⁵ and 10¹⁵ colony-forming units (CFUs) of live attenuated V. cholerae per dosage. In some embodiments, the composition may contain between 10⁵ and 10¹⁵, between 10⁶ and 10¹⁴, between 10⁷ and 10¹³, between 10⁶ and 10⁷ between 10⁸ and 10⁹ total CFUs of live attenuated V. cholerae per dosage. In some embodiments, the composition may contain between 10⁸ and 10⁹ bacterial cells per dosage. In some embodiments, the composition may contain approximately 5×10⁸ total CFUs of V. cholerae per dosage.

In some embodiments, the composition of the invention may comprise between about 0.1 μg/mL-10 mg of cholera toxin such as e.g. the recombinant cholera toxin subunit B (CTB) per dosage. In some embodiments, the composition of the invention may comprise 0.1 μg-5 mg, 0.1 μg-7 mg, 0.1 μg/mL-3 mg, 0.2 μg-4 mg of the recombinant CTB per dosage. In one particular embodiment, the composition of the invention such as the cholera vaccine comprises about 0.75-1.5 mg, preferably 1 mg of the recombinant CTB per dosage.

In a preferred embodiment, the dosage of the dry formulation of V. cholerae vaccine described herein corresponds to the dosage of its liquid formulation. In yet one preferred embodiment, the dosage of the dry cholera vaccine described herein is equal to or about the dosage of the cholera vaccine Dukoral®.

The present invention is capable of other embodiments and of being practiced or of being carried out in various ways. The phraseology and terminology used herein is for the purpose of description and should not be regarded as limiting. The use of “comprising,” “including,” “having,” “containing,” “involving” and variations thereof is meant to encompass the items listed thereafter and equivalents thereof as well as additional items. Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. The methods and techniques of the present disclosure are generally performed according to conventional methods well known in the art and as described in various general and more specific references that are cited and discussed throughout the present specification unless otherwise indicated.

The entire contents of all of the references (including literature references, issued patents, published patent applications, and co-pending patent applications) cited throughout this application are hereby expressly incorporated by reference, in particular for the teaching that is referenced hereinabove. However, the citation of any reference is not intended to be an admission that the reference is prior art.

### EXAMPLES

The following examples are offered to illustrate, but not to limit the claimed invention.

### Example 1: Preparing Dry Formulations of the V. cholerae Vaccine

Materials & Methods

Materials

Dukoral® vaccine suspensions were produced by Valneva Sweden AB (8*1L, Batch #FL00064) and stored at 4° C. Maltodextrin (C*PharmDry 01982, Batch: 02227707) was obtained from Cargill and sucrose (Reag. Ph Eur) from Merck. The glass vials were sterilized in an autoclave prior use.

Sample Preparation

In order to achieve a powder formulation, a mass-ratio of 1:10 of Dukoral® vaccine components and excipients were chosen. The dry content of the pure Dukoral® vaccine was calculated after freeze drying of the vaccine suspension and resulted in 16.4 mg dry material per mL vaccine. Hence, the amounts of the excipients added to the pure Dukoral® suspension prior drying were as stated in Table 1.

TABLE 1
Excipients added to the samples for spray- and freeze-drying.

| Sample | Maltrodextrin:sucrose- | Maltodextrin added per | Sucrose added per mL |
|---|---|---|---|
| acronym | ratio (wt. %) | mL Dukoral ® (mg) | Dukoral ® (mg) |
| A | 100:0 | 164 | 0 |
| B | 90:10 | 147.6 | 16.4 |
| C | 80:20 | 131.2 | 32.8 |

The appropriate amounts of the excipients were added to the pure Dukoral® suspension (typically batch volumes of 400 mL for spray drying and 200 mL for freeze drying were used) and left dissolve for 1 h under magnetic stirring at room temperature prior further use. The prepared suspensions were used the same day for freeze drying or spray drying. Residual samples were stored overnight at +4° C. for visual inspection.

Freeze Drying

Freeze drying of the samples were performed using an Epsilon 2-4 LSCplus (Martin Christ Gmbh, Germany) freeze dryer. The liquid samples (3 mL/vial, equal to 3.2 g resulting in 480±10 mg powder/vial) were first frozen at −40° C. for 4 h on the tempered plate inside the dryer at atmospheric pressure, followed by main drying at 0.1 mbar (equivalent to −42° C. ice sublimation temperature) and +4° C. plate temperature for 16-18 h. Final drying of the samples was performed at 0.004 mbar at +20° C. plate temperature for 4 h. A temperature sensor was immersed in one sample to monitor the drying progress. After completion of the freeze-drying cycle the vials were sealed in air (relative humidity 15-25%) within 10 min to minimize water uptake. In result, glass vials with 480±10 mg per vial were obtained.

Spray Drying

Spray drying was carried out using a two-fluid spray nozzle 1.5 mm in diameter in an in-house built spray drying equipment using the following processing conditions: T_{Inlet}=180° C. and T_{Outlet}=90° C. Flow rate of the feed suspension and drying air was 5 mL/min and approx. 0.8 m3/min, respectively. The spray-dried powders were thereafter divided into glass vials with 480±20 mg per vial. The yield for spray drying was in the range of 65-80%. Hence, approximately 20-35% loss of material due to powder sticking to drying column walls etc.

Total Water Content

Total water content was quantified using thermogravimetric analysis (TGA) using a TGA2 instrument (Mettler Toledo, Switzerland). 2-4 mg powder were placed in an alumina crucible and heated from 25° C. to 250° C. at a rate of 20 K/min. in N2-gas at a flow rate of 5.0 ml/min. The evaluation of the water content (weight loss, %) was done in the interval 40° C. to 125° C. using STARe SW14 software (Mettler Toledo). Samples were measured in duplicates.

Water Activity

The activity of the free water (a_{w}) in the powder samples were characterized by dew point measurement using a Water Activity Meter (AquaLab 4TE). The powder samples were placed in disposable sample cups to cover the bottom. The measurements were carried out at 25° C. Before and after the measurements, verification of a_{w} 0.25 standard was performed. Distillate water has a_{w} 1.

Particle Size

The particle size and size distribution of the pure (liquid) Dukoral® suspension and re-hydrated powders were analysed by laser diffraction using a Mastersizer 3000 instrument (Malvern Panalytical, UK). The refractive index of the dispersant was set to 1.330 and 1.500 for the particles with an absorption index of 0.50. The samples were diluted in Milli Q water and measured in triplicate.

Zeta-Potential

The zeta-potential gives an indication of the surface charge between the Stern and slipping plane layer of particles and was determined by measuring the electrophoretic mobility with a Zetasizer Zen3600 (Malvern Instruments Ltd., U.K.) using the Smoluchowski model.

The Dukoral® suspensions and re-hydrated powders were diluted 10-fold in MilliQ-water and analyzed in disposable measuring cells at 25° C. Each sample was measured in triplicate.

Light Microscopy

The appearance of the pure Dukoral® sample, samples after addition of excipients and of re-hydrated powders were investigated using a Zeiss Axioplan light microscope (Carl Zeiss, Germany) equipped with 20× and 100× magnification objectives.

Results

A summary of pH and zeta-potential of the suspensions, water activity and total water content of the dried formulations (powders) are presented in Table 2. Neither the addition of excipients nor drying procedures had any major effect on the pH of the formulations. The zeta-potential of the bacteria in the pure Dukoral® suspension was −22.0±2.2 mV and was not affected by addition of the excipients (compositions A, B and C). The zeta-potential did not change significantly upon re-hydration of the dried vaccine samples. The total water content in the freeze-dried samples were typically 2.0%, while in the spray dried slightly higher, i.e. from 2.6% to 3.1%. The differences in total water content was also reflected in the water activity (“free” water), showing lower a_{w} values for the freeze-dried samples (0.026±0.003) compared to samples obtained by spray drying (0.10±0.015). These values for water content and water activity are in the range that is common for spray-dried and freeze-dried formulations containing mainly carbohydrates.

TABLE 2
Measured characteristics of the liquid (A, B, C) and dry Dukoral ® formulations.

| | | Aqueous formulations | | | |
|---|---|---|---|---|---|
| | | | Zeta-potential | Dried powder | |
| | | | (m V) | | Total water |
| Sample/ | | Zeta-potential | re-hydrated | | content |
| Date of production | pH | (m V) | powder | a_{w} | (%) |
| Pure Dukoral ® | 7.1 | −22.2 ± 2.2 | — | — | — |
| A | 7.0 | −22.8 ± 0.6 | — | — | — |
| B | 7.0 | −21.9 ± 1.8 | — | — | — |
| C | 7.0 | −22.0 ± 0.9 | — | — | — |
| A-FD* | 7.1 | — | −22.9 ± 1.3 | 0.024 ± 0.000 | 1.9 ± 0.2 |
| B-FD | 7.1 | — | −23.7 ± 2.0 | 0.028 ± 0.004 | 2.0 ± 0.0 |
| C-FD | 7.1 | — | −23.6 ± 2.2 | 0.027 ± 0.004 | 2.0 ± 0.0 |
| A-SD** | 7.0 | — | −21.9 ± 1.7 | 0.096 ± 0.002 | 3.1 ± 0.1 |
| B-SD | 7.0 | — | −21.6 ± 0.9 | 0.095 ± 0.002 | 2.8 ± 0.1 |
| C-SD | 7.0 | — | −23.1 ± 1.2 | 0.108 ± 0.001 | 2.6 ± 0.1 |

*FD = Freeze-dried,
**SD = Spray-dried

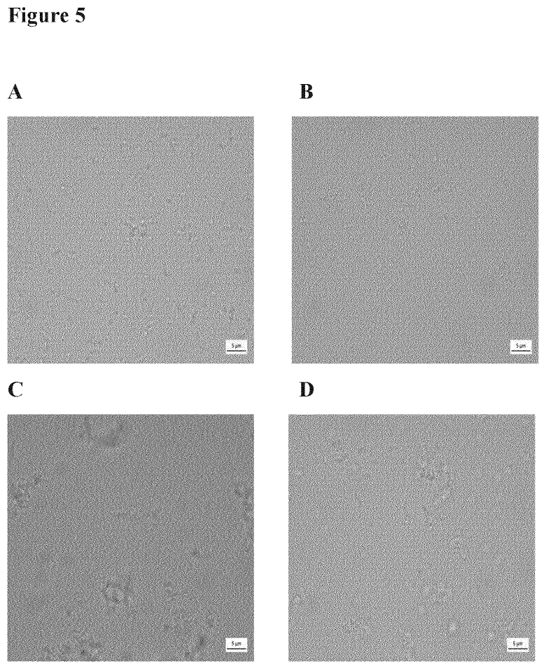

The size and size distribution of the inactivated bacteria and other components in the Dukoral® vaccine were investigated using laser diffraction. Result for the pure Dukoral® suspension and after additions of the excipients at the three compositions A, B and C (see Table 1) are presented in FIG. 1. For the pure Dukoral® sample and compositions with excipients, two distinct peaks were observed around 0.7 μm and 3.0 These peaks do most likely originate from the presence of the inactivated V. cholerae bacteria and was further confirmed by the light microscopy images presented in FIG. 5. The broader peaks in the size range of 10-1000 μm are probably due to aggregates of the bacteria and/or other Dukoral® components, also visible by light microscopy. Light microscopy images acquired at lower magnification (20×) shows presence of solid particles, or aggregates thereof, in the size range of 5-20 μm in all samples (data not shown).

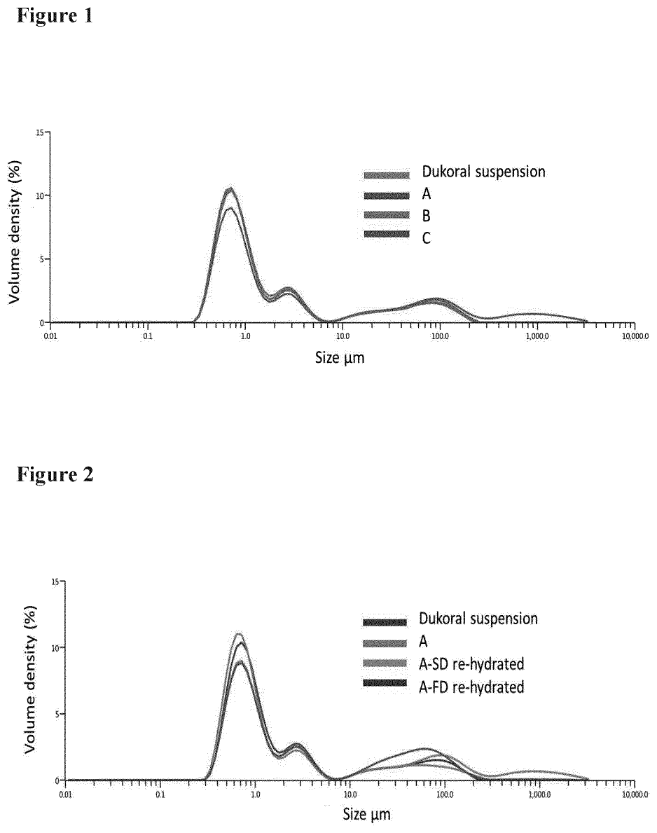

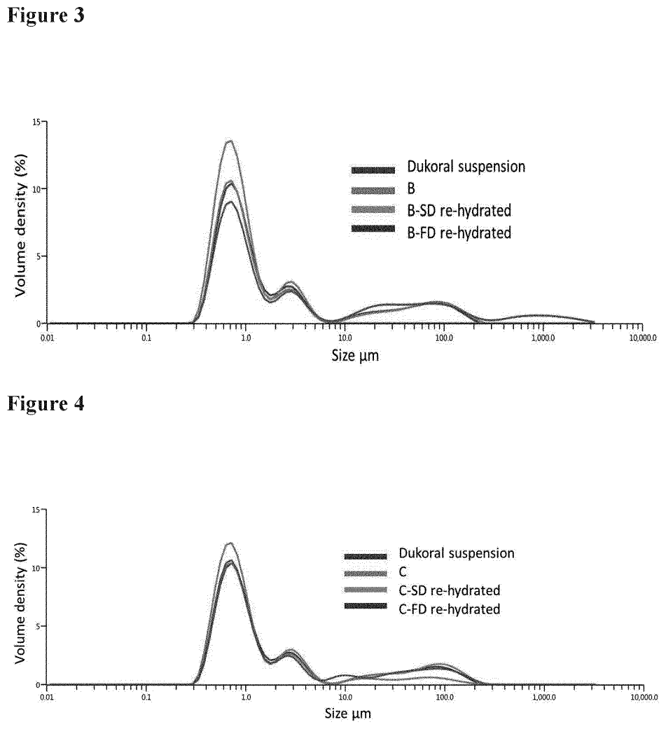

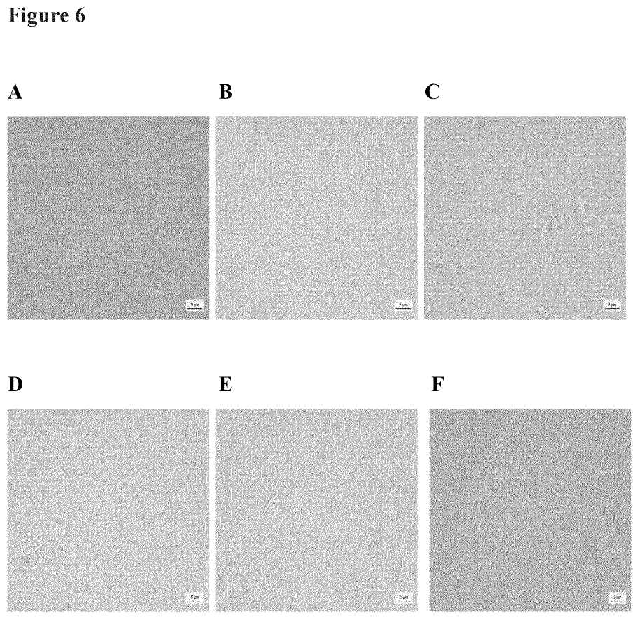

The particle size and size distribution for re-hydrated spray- and freeze-dried formulations did not differ significantly from the pure Dukoral® vaccine or liquid Dukoral® samples with excipients, as displayed in FIG. 2, FIG. 3 and FIG. 4. Light microscopy images of re-hydrated spray- and freeze-dried powders are presented in FIG. 6.

### Example 2: Stability of the Dry Formulations Vs. The Liquid Formulation of V. cholerae Vaccine

Materials & Methods

Materials

Dukoral® vaccine suspension (pure) and dry formulations as described in Example 1.

Sample Preparation

All dry vaccine samples were reconstituted prior to analysis. Reconstitution was carried out by adding 3 ml of Water For Injection (WFI) and gentle vortexing to get homogenous suspension. The dose of 3 ml of the pure Dukoral® vaccine (liquid), which was used for preparing the dry formulations, was used as a reference in all stability studies.

LPS Assay

Measurement of O1 Lipopolysaccharide (LPS) antigen is performed by means of an Enzyme-Linked Immuno Assay (ELISA), developed in-house, which is based on inhibition the LPS antigen present on the surface of V. cholerae bacteria with murine anti-LPS specific antibodies. Test samples are serially diluted and incubated at room temperature with a fixed amount of the monoclonal anti-LPS antibody (produced in-house) in a 96-wells plate blocked with BSA. After antibody binding to bacterial LPS, the inhibition solution is transferred to a 96-wells plate coated with purified LPS. Non-bound monoclonal antibodies present in the samples will bind to LPS immobilized on the microtiter wells. Then, anti-murine antibodies conjugated with peroxidase enzyme (Jackson Laboratories) are added to the wells and the plate is incubated at room temperature. Finally, monoclonal antibodies bound to the wells is visualized by using Ortho Phenylene Diamine (ODP) and hydrogen peroxide as substrates. The amount of monoclonal antibodies bound to the wells is inversely proportional to the amount of bacterial LPS. The enzyme reaction proceeds until the absorbance values at 450 nm of negative control wells (without added inhibitor) reach approximately 1.0. The 50% inhibitory value (ID₅₀) is defined as the dilution of bacterial antigen needed to obtain 50% decrease of absorbance as compared with the control wells with no inhibitor added. In each analysis run, an in-house produced Dukoral vaccine standard and Dukoral vaccine control are used. ID₅₀ for test samples are calculated by regression analysis of the curves by plotting absorbance values against the logarithm of the dilutions of the standard and samples. All calculations are performed in the validated software SoftMax Pro (Molecular Devices, LLC).

Mancini Test

rCTB antigen content in a Dukoral vaccine sample is measured by means of a quantitative Single Radial Immunodiffusion (SRID) method, developed in-house, based on the immunodiffusion method described by Mancini et al. (Mancini G, Carbonara AO, and Heremans J F. 1965. Immunochemical quantitation of antigens by single radial immunodiffusion. Immunochemistry 2: 235-254). Briefly, a 10×10 cm, 1 mm thick, 1.5% Noble agar gel is prepared containing polyclonal antiserum against the rCTB antigen. 5 mm in diameter wells in the gel are prepared by punching and the vaccine sample (10 μl) is added to the well. In each analysis, an in-house produced rCTB standard and rCTB control are added. The immunodiffusion process is carried out at room temperature in the airtight humidity box with humidity about 100%. The equilibrium zone is reached at 24 hours. At that point, the immune complex forms a precipitation ring around the well, which is visualized by Coomassie Blue staining. Measurement of the diameter of the precipitation ring is performed with a Vernier caliper (0.05 mm precision). Within the dynamic range of the method, the area within the ring is directly proportional to the concentration of rCTB added to the well. Measurement of rCTB concentration in unknown samples containing rCTB is determined by interpolation to linear regression curve for the standard.

Stability Study

The stability study includes three arms corresponding to the following storage conditions:
5° C.±3° C.; ambient humidity
25° C.±2° C.; 60±5% RH,
40° C.±2° C.; 75±5% RH,
   wherein RH is the relative humidity. These are fixed intervals defined by the ICH to correlate with different climatic zones: zone II is 25° C.±2° C. and 60±5%, while zone IV is 30±2° C. and or 75±5% RH. We preferably aim at climatic zone IV as our worst case (important for e.g. catastrophic event in countries where cholera is endemic). The stability will be monitored up to 3 years. Samples are pull out at 0, 6, 12, 24 and/or 36 months. Vaccine stability is evaluated by either LPS assay or Mancini test, or both.

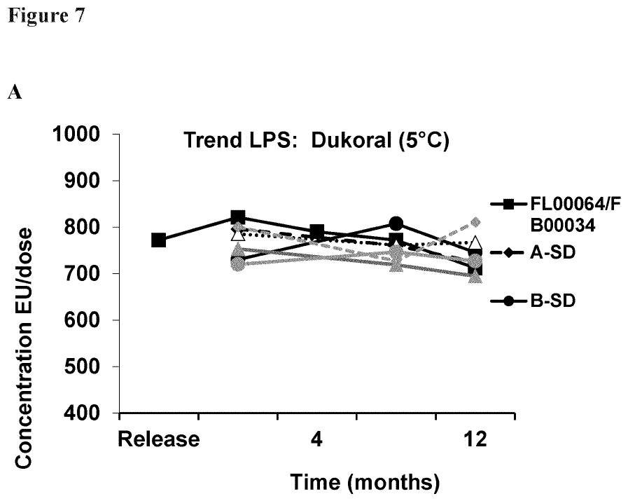

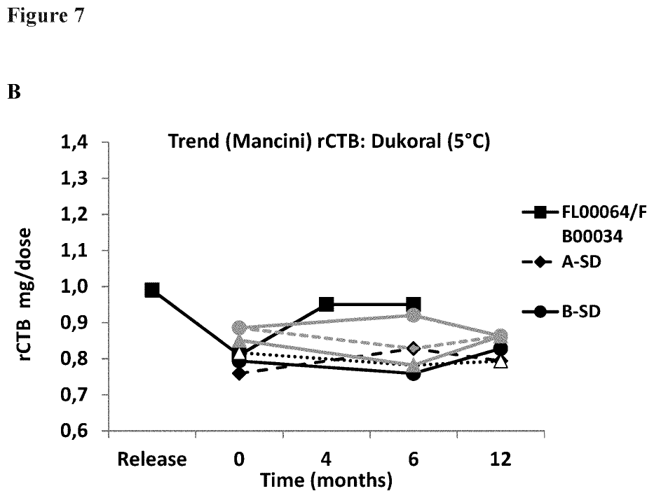

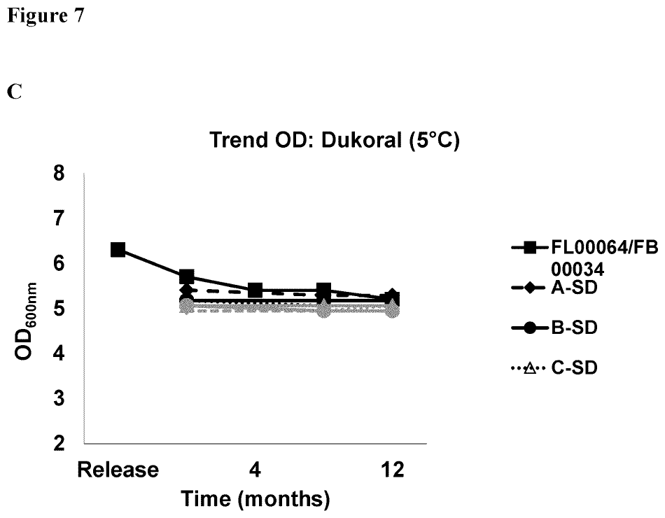

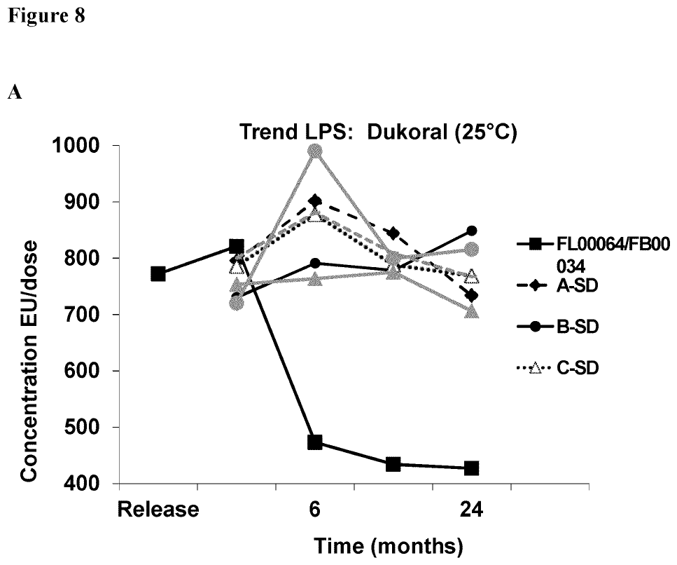

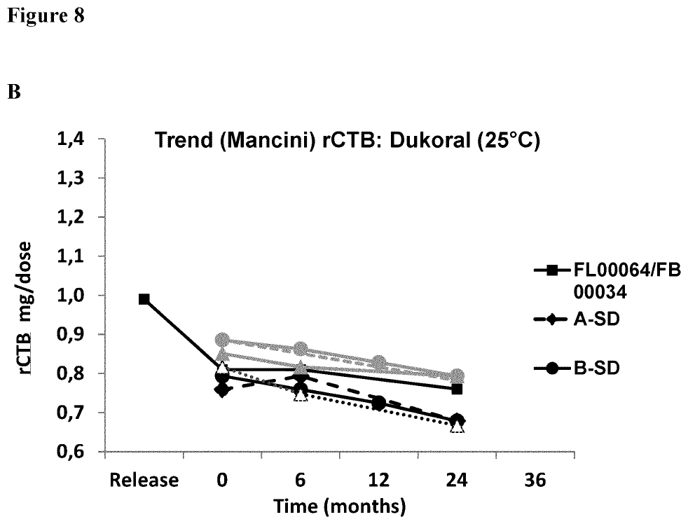

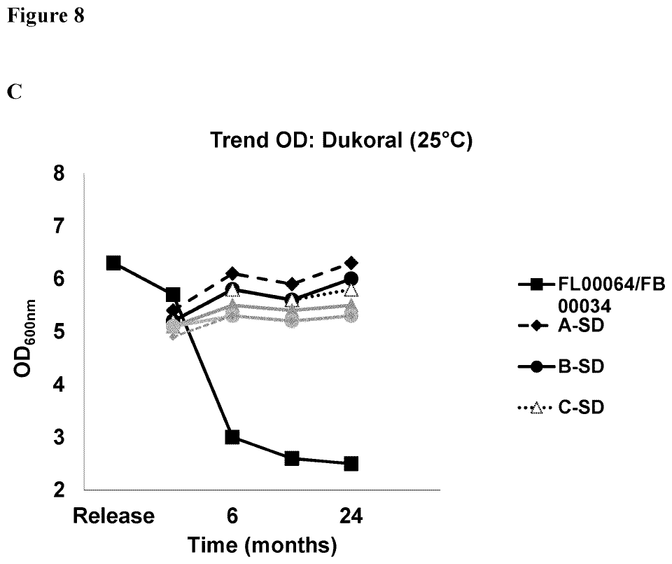

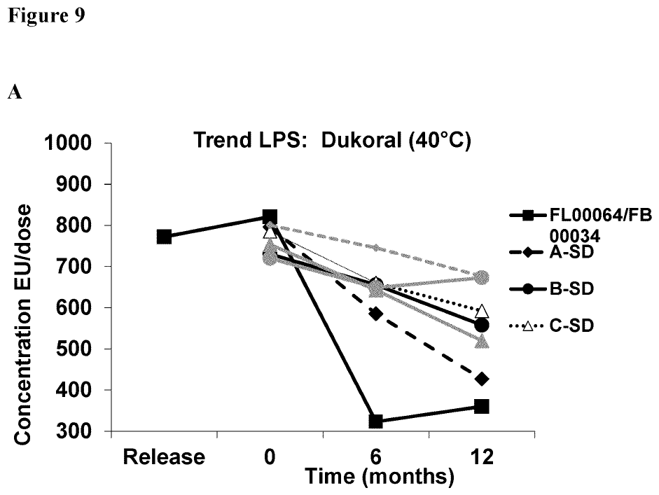

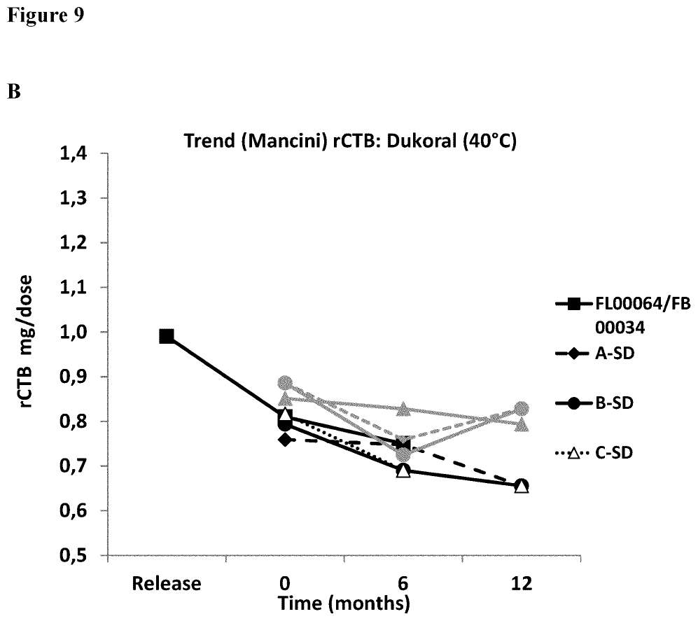

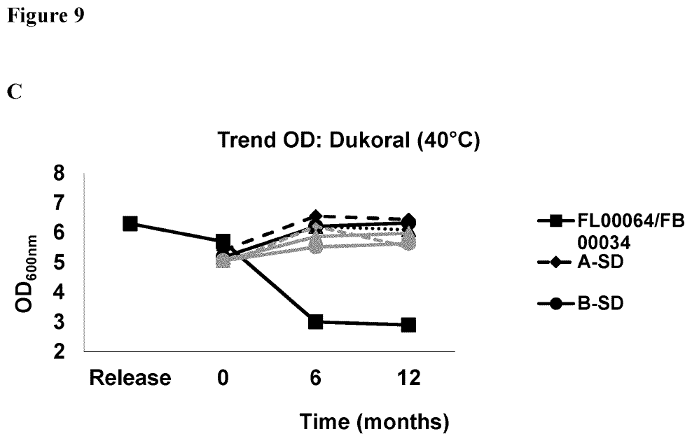

Results of the stability study are shown in Tables 3 to 11 and FIGS. 7 to 9.

TABLE 3
Stability at 5° C., LPS data

| | | LPS (EU/Dose)* | | | |
|---|---|---|---|---|---|
| | Release | t = | t = | t = | t = |
| Formulation | value ** | 0 mo*** | 4 mo | 6 mo | 12 mo |
| Reference (Oral | 772 | 821 | 790 | 772 | 712 |
| solution) | | | | | |
| A-SD | | 796 | n.p.**** | 760 | 725 |
| B-SD | | 730 | | 807 | 745 |
| C-SD | | 785 | | 761 | 767 |
| A-FD | | 753 | | 719 | 695 |
| B-FD | | 720 | | 746 | 727 |
| C-FD | | 800 | | 727 | 811 |

*ELISA Units/dose of 3 ml
**Release value is the result after production of the oral vaccine. The dry formulations were released at t = 0
***mo = months from start of stability study
****n.p. = not performed at this time point

TABLE 4
Stability at 5° C., rCTB data

| | | rCTB (mg/Dose) | | | |
|---|---|---|---|---|---|
| | Release | t = | t = | t = | t = |
| Formulation | value* | 0 mo** | 4 mo | 6 mo | 12 mo |
| Reference (Oral | 0.99 | 0.81 | 0.95 | 0.95 | n.p. |
| solution) | | | | | |
| A-SD | | 0.76 | n.p.*** | 0.83 | 0.79 |
| B-SD | | 0.79 | | 0.76 | 0.83 |
| C-SD | | 0.82 | | 0.78 | 0.79 |
| A-FD | | 0.85 | | 0.78 | 0.86 |
| B-FD | | 0.89 | | 0.92 | 0.86 |
| C-FD | | 0.89 | | 0.83 | 0.86 |

*Release value is the result after production of the oral vaccine. The dry formulations were released at t = 0
**mo = months from start of stability study
***n.p. = not performed at this time point

TABLE 5
Stability at 5° C.; Optical Density data

| | | OD 600ₙₘ* | | | |
|---|---|---|---|---|---|
| | Release | t = | t = | t = | t = |
| Formulation | value ** | 0 mo*** | 4 mo | 6 mo | 12 mo |
| Reference (Oral | 6.3 | 5.7 | 5.4 | 5.4 | 5.2 |
| solution) | | | | | |
| A-SD | | 5.4 | n.p.**** | 5.3 | 5.3 |
| B-SD | | 5.2 | | 5.2 | 5.2 |
| C-SD | | 5.2 | | 5.1 | 5.1 |
| A-FD | | 5.1 | | 5.1 | 5.1 |
| B-FD | | 5.1 | | 4.9 | 4.9 |
| C-FD | | 4.9 | | 4.9 | 5.1 |

*Optical Density measured as Absorbance at 600 nm wavelength
**Release value is the result after production of the oral vaccine. The dry formulations were released at t = 0
***mo = months from start of stability study
****n.p. = not performed at this time point

TABLE 6
Stability at 25° C.; LPS data

| | | LPS (EU/Dose)* | | | |
|---|---|---|---|---|---|
| | Release | t = | t = | t = | t = |
| Formulation | value ** | 0 mo*** | 6 mo | 12 mo | 24 mo |
| Reference (Oral | 772 | 821 | 473 | 434 | 491 |
| solution) | | | | | |
| A-SD | | 796 | 902 | 844 | 734 |
| B-SD | | 730 | 791 | 779 | 849 |
| C-SD | | 785 | 877 | 788 | 768 |
| A-FD | | 753 | 764 | 775 | 706 |
| B-FD | | 720 | 990 | 799 | 815 |
| C-FD | | 800 | 881 | 808 | 767 |

*ELISA Units/dose of 3 ml
**Release value is the result after production of the oral vaccine. The dry formulations were released at t = 0
***mo = months from start of stability study

TABLE 7
Stability at 25° C.; rCTB data

| | | rCTB (mg/Dose) | | | |
|---|---|---|---|---|---|
| | Release | t = | t = | t = | t = |
| Formulation | value* | 0 mo** | 6 mo | 12 mo | 24 mo |
| Reference (Oral | 0.99 | 0.81 | 0.81 | n.p.*** | 0.87 |
| solution) | | | | | |
| A-SD | | 0.76 | 0.79 | n.p. | 0.68 |
| B-SD | | 0.79 | 0.76 | 0.72 | 0.68 |
| C-SD | | 0.82 | 0.75 | n.p. | 0.67 |
| A-FD | | 0.85 | 0.82 | n.p. | 0.79 |
| B-FD | | 0.89 | 0.86 | 0.83 | 0.79 |
| C-FD | | 0.89 | 0.85 | n.p. | 0.78 |

*Release value is the result after production of the oral vaccine. The dry formulations were released at t=0
**mo = months from start of stability study
***n.p. = not performed at this time point

TABLE 8
Stability at 25° C.; Optical Density data

| | | OD 600ₙₘ* | | | |
|---|---|---|---|---|---|
| | Release | t = | t = | t = | t = |
| Formulation | value** | 0 mo*** | 6 mo | 12 mo | 24 mo |
| Reference (Oral | 6.3 | 5.7 | 3.0 | 2.6 | 2.9 |
| solution) | | | | | |
| A-SD | | 5.4 | 6.1 | 5.9 | 6.3 |
| B-SD | | 5.2 | 5.8 | 5.6 | 6.0 |
| C-SD | | 5.2 | 5.8 | 5.6 | 5.8 |
| A-FD | | 5.1 | 5.5 | 5.4 | 5.5 |
| B-FD | | 5.1 | 5.3 | 5.2 | 5.3 |
| C-FD | | 4.9 | 5.3 | 5.2 | 5.3 |

*Optical Density measured as Absorbance at 600 nm wavelength
**Release value is the result after production of the oral vaccine. The dry formulations were released at t = 0
***mo = months from start of stability study

TABLE 9
Stability at 40° C.; LPS data

| | | LPS (EU/Dose)* | | |
|---|---|---|---|---|
| | Release | t = | t = | t = |
| Formulation | value** | 0 mo*** | 6 mo | 12 mo |
| Reference (Oral | 772 | 821 | 323 | 360 |
| solution) | | | | |
| A-SD | | 796 | 585 | 427 |
| B-SD | | 730 | 656 | 558 |
| C-SD | | 785 | 660 | 592 |
| A-FD | | 753 | 643 | 520 |
| B-FD | | 720 | 649 | 673 |
| C-FD | | 800 | 745 | 676 |

*ELISA Units/dose of 3 ml
**Release value is the result after production of the oral vaccine. The dry formulations were released at t = 0
***mo = months from start of stability study

TABLE 10
Stability at 40° C.; rCTB data

| | | rCTB (mg/Dose) | | |
|---|---|---|---|---|
| | Release | t = | t = | t = |
| Formulation | value* | 0 mo** | 6 mo | 12 mo |
| Reference (Oral | 0.99 | 0.81 | 0.75 | n.p.*** |
| solution) | | | | |
| A-SD | | 0.76 | 0.75 | 0.66 |
| B-SD | | 0.79 | 0.69 | 0.66 |
| C-SD | | 0.82 | 0.69 | 0.66 |
| A-FD | | 0.85 | 0.83 | 0.79 |
| B-FD | | 0.89 | 0.72 | 0.83 |
| C-FD | | 0.89 | 0.76 | 0.83 |

*Release value is the result after production of the oral vaccine. The dry formulations were released at t = 0
**mo = months from start of stability study
***n.p. = not performed at this time point

TABLE 11
Stability at 40° C., Optical Density data

| | | OD 600ₙₘ* | | |
|---|---|---|---|---|
| | Release | t = | t = | t = |
| Formulation | value** | 0 mo*** | 6 mo | 12 mo |
| Reference (Oral | 6.3 | 5.7 | 3.0 | 2.9 |
| solution) | | | | |
| A-SD | | 5.4 | 6.6 | 6.4 |
| B-SD | | 5.2 | 6.2 | 6.3 |
| C-SD | | 5.2 | 6.2 | 6.1 |
| A-FD | | 5.1 | 5.9 | 6.0 |
| B-FD | | 5.1 | 5.5 | 5.6 |
| C-FD | | 4.9 | 6.2 | 5.5 |

*Optical Density measured as Absorbance at 600 nm wavelength
**Release value is the result after production of the oral vaccine. The dry formulations were released at t = 0
***mo = months from start of stability study

What is claimed is:

1. Use of a pharmaceutical composition comprising an inactivated or attenuated whole cell bacteria, wherein said composition has a water activity of less than or equal 0.15, and wherein said inactivated or attenuated whole cell bacteria within the composition does not deviate more than +/−50% in their potency for at least one year when stored at a temperature between about 20° C. to 40° C.

2. Use of a pharmaceutical composition comprising an inactivated or attenuated whole cell bacteria, said composition has a water activity of less than or equal 0.15, for counteracting decrease of potency of said inactivated or attenuated whole cell bacteria that occurs during storage of said composition for at least one year at a temperature between about 20° C. and 40° C., as compared to decrease of potency of the inactivated or attenuated whole cell bacteria within a pharmaceutical composition that has water activity more than 0.15 when stored under the same conditions.

3. Use of a pharmaceutical composition comprising an inactivated or attenuated whole-cell bacteria, said composition has a water activity of less than or equal 0.15, for prolonging a storage life of said inactivated or attenuated whole cell bacteria within said composition to at least one year at a temperature between about 20° C. and 40° C., preferably at about 25° C. and relative humidity 60±5%, as compared to a storage life of the inactivated or attenuated whole cell bacteria within a pharmaceutical composition that has a water activity of more than 0.15 when stored under the same condition.

4. Use of a pharmaceutical composition comprising an inactivated or attenuated whole-cell bacteria, said composition has a water activity of less than or equal 0.15, for prolonging a storage life of said inactivated or attenuated whole cell bacteria within said composition to at least one year at a temperature between about 25° C. and 35° C., preferably at about 30° C. and relative humidity 75±5%, as compared to a storage life of said inactivated or attenuated whole cell bacteria within a pharmaceutical composition that has a water activity of more than 0.15 when stored under the same conditions.

5. Use of the pharmaceutical composition according to claim 1 or 2, wherein potency of said pharmaceutical composition is measured by an LPS-ELISA.

6. Use of the pharmaceutical composition according to claims 1 to 5, wherein said bacteria is selected from the group consisting of Vibrio cholerae, Clostridium difficile, Clostridium perfringens, Clostridium botulinum, Clostridium tetani, Corynebacterium diphtheria, Shigella dysentheriae, Staphylococcus aureus, Pseudomonas aeruginosa, Bordetella pertussis, Bacillus anthracis, Escherichia coli, preferably Vibrio cholerae.

7. Use of the pharmaceutical composition according to claims 1 to 6, wherein said composition further comprises at least one recombinant toxin.

8. Use of the pharmaceutical composition according to claim 7, wherein the toxin is selected from the group consisting of cholera toxin (CT) or its B subunit (CTB), staphylococcal toxins, diphtheria toxin, tetanus toxin, pertussis toxin, shiga toxin, shiga-like toxin, botulinum neurotoxin, Clostridium difficile toxins, Clostridium perfringens alpha toxin, Bacillus anthracis toxin, Pseudomonas aeruginosa alpha toxin, heat-labile enterotoxin (LT) of enterotoxigenic Escherichia coli (ETEC) and heat-stable enterotoxin (ST) of enterotoxigenic Escherichia coli (ETEC), preferably cholera toxin (CT) or its B subunit (CTB).

9. Use of the pharmaceutical composition according to any preceding claim, wherein said composition has a water activity of less than or equal 0.1.

10. Use of the pharmaceutical composition according to any preceding claim, wherein said composition has a total water content about or less than 3%, preferably between about 3% and 2%.

11. Use of the pharmaceutical composition according to any preceding claim, wherein said composition comprises a stabilizer.

12. Use of the pharmaceutical composition according to any preceding claim, wherein said composition is stored or has the ability to be stored at a temperature between about 20° C. and 40° C., preferably at about 25° C. or 30° C., for at least two or three years.

13. Use of the pharmaceutical composition according to any preceding claim, wherein said composition is formulated as a powder, tablet, granule or capsule.

14. Use of the pharmaceutical composition according to any preceding claim, wherein said composition is a vaccine.

15. A vaccine comprising an inactivated whole-cell bacteria V. cholerae, wherein said vaccine has a water activity of less than or equal 0.15 and storage life of at least one year at a temperature between about 20° C. and 40° C., preferably at about 25° C. or 30° C.

16. A vaccine comprising an inactivated whole-cell bacteria V. cholerae, wherein said vaccine has a water activity of less than or equal 0.15 and storage life of at least one year at a temperature between about 20° C. and 40° C., preferably at about 25° C. or 30° C., and wherein vaccine potency does not deviate more than +/−50% during storage at said temperature.

17. The vaccine according to claim 15 or 16, wherein V. cholerae bacteria are of at least one of the following strains Vibrio cholerae O1 Inaba classical biotype, Vibrio cholerae O1 Inaba El Tor biotype, Vibrio cholerae O1 Ogawa classical biotype, or combinations thereof.

18. The vaccine according to claims 15 to 17, wherein a total amount of V. cholerae bacteria is between 1.0×10¹¹ and 1.5×10¹¹, preferably 1.25×10¹¹ per dose.

19. The vaccine according to claims 15 to 18, wherein said vaccine further comprises a recombinant cholera toxin (CT) or its B subunit (CTB).

20. The vaccine according to claim 19, wherein the recombinant CT or CTB is present at the amount from about 0.75 to 1.25 mg, preferably 1.0 mg per dose.

21. The vaccine according to claims 15 to 20, wherein said vaccine further comprises a pharmaceutically acceptable carrier and/or excipient, selected from an adjuvant, buffer, preservative, stabilizer, surfactant, flavor, used either alone or in combination.

22. The vaccine according to claims 15 to 21, wherein the vaccine further comprises the stabilizer selected from the group consisting of sucrose, tregalose, raffinose, lactose, maltose, mannitol, sorbitol, maltodextrin, arginine, histidine, glycine, used either alone or in variable combinations.

23. A vaccine comprising per dose between 1.0×10¹¹ and 1.5×10¹¹, preferably 1.25×10¹¹ total amount of bacteria of the following strains:
Vibrio cholerae O1 Inaba, classical biotype (heat inactivated)
Vibrio cholerae O1 Inaba, El Tor biotype (formalin inactivated)
Vibrio cholerae O1 Ogawa, classical biotype (heat inactivated)
Vibrio cholerae O1 Ogawa, classical biotype (formalin inactivated),
excipients: sodium dihydrogen phosphate dihydrate (2.0 mg), disodium hydrogen phosphate dihydrate (9.4 mg) and sodium chloride (26 mg),
further comprising a stabilizer,
wherein said vaccine has a water activity of less than or equal 0.15 and a storage life of at least one year at a temperature between about 20° C. and 40° C., preferably at 25° C. or 30° C., and wherein vaccine potency does not deviate more than +/−50% during storage at said temperature.

24. A vaccine comprising per dose between 1.0×10¹¹ and 1.5×10¹¹, preferably 1.25×10¹¹ total amount of bacteria of the following strains:
Vibrio cholerae O1 Inaba, classical biotype (heat inactivated)
Vibrio cholerae O1 Inaba, El Tor biotype (formalin inactivated)
Vibrio cholerae O1 Ogawa, classical biotype (heat inactivated)
Vibrio cholerae O1 Ogawa, classical biotype (formalin inactivated),
a recombinant cholera toxin B subunit (CTB) (0.75-1.25 mg, preferably 1 mg),
excipients: sodium dihydrogen phosphate monohydrate (2.0 mg), disodium hydrogen phosphate dihydrate (9.4 mg) and sodium chloride (26 mg),
further comprising a stabilizer,
wherein said vaccine has a water activity of less than or equal 0.15 and a storage life of at least one year at a temperature between about 20° C. and 40° C., preferably at 25° C. or 30° C., and wherein vaccine potency does not deviate more than +/−50% during storage at said temperature.

25. The vaccine according to claim 23 or 24, wherein the ratio between dry vaccine content and the stabilizer is 1:10 (w/w).

26. The vaccine according to claims 23 to 25, wherein the stabilizer is maltodextrin or sucrose, used along or combined in the ratio 9:1 (w/w) or 4:1 (w/w).

27. The vaccine according to claim 26, wherein the vaccine comprises about 164 mg/mL of maltodextrin, or about 148 mg/mL of maltodextrin and about 16 mg/mL of sucrose, or about 132 mg/mL of maltodextrin and about 32 mg/mL of sucrose.

28. The vaccine according to any of claims 15 to 27, wherein said vaccine has the water activity of less than or equal to 0.1, preferably between 0.1 and 0.02.

29. The vaccine according to any of claims 15 to 28, wherein said vaccine has the water activity about 0.03.

30. The vaccine according to any of claims 15 to 29, wherein said vaccine has a total water content about or less than 3%, preferably between 3% and 2%.

31. The vaccine according to any of claims 15 to 30, wherein the immunogenicity of said vaccine remains stable for at least two years during storage at a temperature between about 20° C. and 40° C.

32. The vaccine according to any of claims 15 to 31, wherein the immunogenicity of said vaccine remains stable for more than two years during storage at a temperature between about 20° C. and 40° C.

33. The vaccine according to any of claims 15 to 32, wherein said vaccine is freeze-dried.

34. The vaccine according to any of claims 15 to 32, wherein said vaccine is spray-dried.

35. The vaccine according to any of claims 15 to 34, wherein said vaccine is formulated as a powder, tablet, granule or capsule.

36. The vaccine according to any of claims 15 to 35 for the prevention and/or treatment of V. cholerae infection and/or cholera disease.

37. A method of prevention and/or treatment of V. cholerae infection and/or cholera disease comprising administration to a subject of need a sufficient amount of the vaccine according to any of claims 15 to 36.

38. A process for producing the composition according to any of claims 1 to 13 or the vaccine according to any of claims 15 to 36, wherein said process comprises the following steps: i) mixing of relevant ingredients, ii) freeze drying, and iii) dosage formation.

39. A process for producing composition according to any claims 1 to 13 or the vaccine according to any of claims 15 to 36, wherein said process comprises the following steps: i) mixing of relevant ingredients, ii) spray drying, and iii) dosage formation.
